(19) United States
(12) Patent Application Publication
(10) Pub. No.: US 2005/0234309 A1
(43) Pub. Date: Oct. 20, 2005

(54) Method and apparatus for classification of movement states in Parkinson's disease

(76) Inventor: David Klapper, Miami Beach, FL (US)

Correspondence Address: EPSTEIN DRANGEL BAZERMAN & JAMES, LLP, 60 EAST 42ND STREET, SUITE 820, NEW YORK, NY, 10165, US

(21) Appl. No.: 11/030,490

(22) Filed: Jan. 5, 2005

Related U.S. Application Data
(60) Provisional application No. 60/534,797, filed on Jan. 7, 2004.

(51) Int. Cl. A61B 5/00

(52) U.S. Cl. 600/300

(57) ABSTRACT

For Parkinson's patients to function at their best, their medications need to be optimally adjusted to the diurnal variation of symptoms. For this to occur, it is important for the managing clinician to have an accurate picture of how the patient's bradykinesia/hypokinesia and dyskinesia and the patient's perception of movement state fluctuate throughout the normal daily activities. The present invention uses wearable accelerometers coupled with computer implemented learning and statistical analysis techniques in order to classify the movement states of Parkinson's patients and to provide a timeline of how the patients fluctuate throughout the day.

The present invention relates to a method and apparatus for the classification of movement states in patients with Parkinson's disease and more particularly, to a system in which sensor apparatus worn by the patient to monitor movement over time provides information to a computer for use in a previously developed prediction algorithm for classifying the movement states of the patient to assist the managing clinician in determining the timing and dosing of medications to maximize patient function.

### BACKGROUND OF THE INVENTION

1. Field of the Invention

Parkinson's disease is a common disorder affecting at least 750,000 to one million Americans. Parkinson's disease causes progressive difficulty in moving. This includes slowness of movement, decreased amount of movement and difficulty initiating movement. Oftentimes there is an associated tremor as well as balance and posture problems. These difficulties with movement can be quite debilitating for patients. They seriously affect Parkinson's patients' quality of life as well as their ability to perform necessary activities of daily living.

Parkinson's disease is, however, a treatable condition. Medications can improve the debilitating decrease of movement. Unfortunately, medications often cause serious side effects such as abnormal movements and/or abnormal posturing (e.g. chorea and dystonia) that are in and of themselves debilitating. The effectiveness of the medication as well as the side effects of the medication is related to the concentration of the medication in the patient's brain. For instance, too low a concentration may not relieve the Parkinson's symptoms and too high a level may lead to the abnormal movements. As a patient's disease gets worse over time, medications become less effective and the effect of each dose lasts less. In addition, abnormal movements, that are side effects of medication, tend to increase. For these reasons, patients who have had Parkinson's disease for several years are often in a delicate balance between the benefit of medication and the side effects of medication. Even a slight change of dosing or timing of a patient's Parkinson's medication may have profound effects on how that patient is able to function.

In order to maintain this delicate balance of medication, the managing clinician needs to have accurate and reliable information about how the patient's movement changes throughout the day. Slow and decreased movements at a particular time of day may lead the clinician to increase medication at that time. Abnormal movements such as chorea and dystonia may require different types of medication adjustments depending on the timing and type of abnormal movement. Other abnormalities such as freezing or rapid fluctuations (i.e. “on-off”) require their own interventions.

Unfortunately, a clinician who sees a Parkinson's patient in the office is only able to witness the patient at a single point in time and has no observational information about the patient's daily fluctuations at home. Clearly, the history given by the patient is very useful, but it is prone to recollection errors as well as many patients' difficulty in judging precisely what sort of abnormal or impaired movements they where having during the course of the day. Impaired cognitive function is common in Parkinson's patients and may make getting an accurate and reliable history even harder. Having a patient take a diary can be helpful. However, patient self-reporting diaries are difficult to comply with (often not completely filled out) and also suffer from the same problems that can cause inaccurate histories.

Accordingly, clinicians who care for Parkinson's patients, if provided with detailed information as to the movement states of their patient over time should be able to effectively manage and offset the hour-by-hour fluctuations in movement that these patients often experience. That could be achieved if the symptoms of Parkinsonism such as bradykinesia, hypokinesia and akinesia, and medication-related side effects such as dyskinesia could be reported to the clinician in a manner that accurately conveys the timing and severity of the symptoms. The clinician could then tightly adjust and titrate the timing and dosing of medication, allowing the patient to function at his or her best.

Patient history and patient self-reporting diaries are currently used for this purpose, but they have problems related with patients' compliance, completeness and reliability. A monitor that could be worn by the patient while he or she is at home and could issue to the clinician a report of how the patient has been moving over the course of the day would be of great help.

Parkinsonism is defined as two of the following: tremor at rest, rigidity, slow and decreased movements, flexed posture, loss of proper postural stabilizing reflexes and episodes of sudden inability to move with at least one being tremor or slow movement.

Most medications for Parkinson's disease work by compensating for the progressive loss of dopamine secreting neurons that is the cause for this disease. These include levodopa (a precursor of dopamine), dopamine agonists, medications that inhibit the breakdown of dopamine and anticholinergics. Anticholinergics are thought to work by rebalancing the dopamine/acetylcholine balance in the brain that was offset by the loss of dopamine neurons. They are not a first line medication, but are used mostly in treating tremor. Levodopa is the standard medication and typically the most effective. However, many abnormal movements have been attributed to levodopa therapy and there is some thought that it may be toxic to the dopamine secreting neurons and may therefore make the disease worse. For this reason, some patients are started on other Parkinson's medications first. Still, in the end, patients typically end up on levodopa, usually in addition to other medications.

In addition to medication, there are neurosurgical procedures that can be used to treat Parkinson's disease. These include selectively ablative procedures (e.g. pallidotomy, thaladotomy), electrical stimulator implantation and cell transplants. These procedures have their specific indications, but are generally performed in more advanced disease and typically do not eliminate the need for medication.

When a patient has decreased movement, he/she is said to be in the “off” state. Normal or increased movement is said to be “on”. Oftentimes patients gradually turn “off” as the effect of medication wears off (“wearing off phenomenon”); sometimes the switch from “on” to “off” may be more abrupt (“on-off phenomenon” sometimes called “yo-yoing”) and may rapidly switch back and forth from “on” to “off”. This is usually a sign of advanced disease. Collectively, wearing off and yo-yoing are termed “response fluctuations”.

Decreased movement in the “off” state can be in the form of slow movement (“bradykinesia”), paucity of movement (“hypokinesia”) or difficulty initiating movement (“akinesia”). “Freezing”, a sudden inability to move, may occur in the “off” state or the “on” state, with different clinical ramifications (“off” state freezing may improve with more medication, but “on” state freezing is more problematic).

“Dyskinesia” is a general term for abnormal movements (other than tremor). There are many subtypes of dyskinesia. The two most relevant ones for our purposes are chorea (abnormal arrhythmic jerky movements) and dystonia (abnormal posturing). These are typically felt to be side effects of medication.

Both dyskinesia and response fluctuation are quite frequent in Parkinson's patients. One community based study found that of the 70% of Parkinson's disease patients treated with levodopa, 40% had response fluctuations and 28% had dyskinesias. According to that study, after only 18 months of treatment with levodopa (relatively early in disease course) 51% developed wearing off, 5% the more severe “on-off” fluctuations and 26% developed dyskinesias. These problems become more severe with the duration of disease. The symptoms tend to progress with increasing duration of disease and therefore the trend to increasing longevity will likely increase the prevalence of dyskinesias and response fluctuations even beyond where they stand.

Parkinson's research relies on the ability of investigators to compare the Parkinsonism (or dyskinesia) of one patient with that of another (or to compare the same patient at two different time points). For this reason, there is much literature on ways to quantify (i.e. score) patients' degree of Parkinsonism (or dyskinesia). The methods that are most commonly used to quantify a Parkinson's patient's state are observational rating scales and patient self report diaries. Device-based monitoring, both in laboratory and ambulatory, has also been used.

The present invention was developed to provide information that is more useful to the clinician in practical clinical terms than the information that one is likely to be able to obtain in the real world through the use of patient diaries or clinical observation. In clinical practice, diaries are often poorly done because it is hard to get patients to fill out the diaries well, even if the patients are cognitively intact. Patients that have cognitive impairment are even less able to perform this task well. Further, observational scores over extended periods of time are of course not feasible in clinical use. It is unlikely that the clinician will spend 24 hours at home with a patient, taking notes on the patient's condition throughout the period. Thus, as a practical matter, it is believed that the present invention will provide a more accurate assessment of how the movement states of the patient change over the course of time.

As will be explained in detail below, observational rating scales and patient self-reporting diaries of symptoms are used in my invention to create the classifier. To build the classifier, accelerometric recordings of Parkinson's patients and corresponding clinical annotations as to their state of movement (e.g. degree of dyskinesia, bradykinesia/hypokinesia) are required. Those recordings and clinical annotations are used to train the classification algorithm. Observational rating scales and patient self-reporting diaries could be used for this annotation.

As opposed to patient self reporting diaries, observational rating scales are typically used by professionals who are observing the patient and not by the patient himself/herself In contrast to self-reporting diaries, such rating scales do not reflect how the movement state is experienced by the patient (e.g. if the movement state is bothersome or not).

There are rating scales for the staging of Parkinson's as well as for the momentary level of Parkinsonism or dyskinesia. Since the staging of Parkinson's is only relevant to this invention as a means to stratify patients, the focus is on the momentary scales.

The simplest rating scale is a “continuum” scale. These scales typically treat tremor and bradykinesia (which occur in the “off” state) as the polar opposite of, dyskinesia (which tends to occur in the “on” state). Such a scale may give a negative integer for an “off” state, zero for “on” with no dyskinesia and a positive value for “on” with dyskinesia. This is appealing since the whole rating of the patient is encapsulated in a single number. In addition, the rating is simple to do and can be repeated as often as one needs (since it is an instantaneous measure).

There are however several drawbacks. Firstly, dyskinesia and “off” are not truly opposites. Some dyskinesia can occur in the “off” state. Furthermore, different parts of the body may be in different states at the same time. For these reasons, such rating scales not commonly used in clinical investigations. Still, it should be noted that patient self-reporting diaries typically do rate “off”—“on”/dyskinetic as a single dimension, similar to these continuum scales, and patient self reporting diaries are likely the most useful clinical measure for management of patients.

It would probably not be advisable to use a continuum scale as the basis of how the clinician/observer scores how the subjects in the project are doing. Since such scales are not validated and are not commonly used, a device that can only produce output in terms of an observational continuum scale would likely have difficulty being approved and being accepted by clinicians. This stands in contrast to diaries that are completed by the patient (not the clinician). These are typically done on a continuum scale, but are generally accepted.

The AIMS (Abnormal Involuntary Movements Scale) scale for assessing dyskinesia is often used in clinical studies. It was developed originally for assessment of tardive dyskinesia (not Parkinsonism). Therefore it has a strong emphasis on oral and facial dyskinesias which are common in tardive dyskinesia, but are not common in Parkinsonism. For this reason, Parkinson's investigators will often modify the AIMS by leaving out the oral/facial parts of the scale. The scale uses 0-4 ratings which are simple and can be repeated every 15 minutes or so. However, there are no clear descriptions (anchors) that would tell an observer what each number on the 0-4 scale signifies. AIMS includes assessment of the trunk the arms and the legs and includes both observer and patient ratings of severity. It has not been psychometrically tested for Parkinson's patients.

The modified AIMS scale would be a useful method to clinically annotate for this project. It is commonly used and accepted and using it would likely make the device easier to be approved for clinical use and more likely to be accepted. The modified AIMS scale does have somewhat more detail than is currently used for clinical purposes (e.g. separate subscales for upper extremities, lower extremities and trunk). Still, this added information may potentially be useful to clinicians.

The Dyskinesia Subjective Rating Scale is another scale used in measuring dyskinesia. It is not suitable as an instantaneous measure however because it is based on history taken from the patient about how the dyskinesia affects various activities.

The UPDRS motor score (based on a subsection of UPDRS) has been used as a means to score bradykinesia/hypokinesia. However, a poor correlation between the UPDRS motor score and activity counts (suprathreshold accelerations) was found, implying that hypokinesia is poorly represented by the UPDRS scoring scheme.

A single question from the UPDRS examination entitled “body bradykinesia/hypokinesia” has also been used. It does not require an active examination (as does much of the rest of the UPDRS motor exam). Therefore, it would be suitable for the continual, unobtrusive scoring that is necessary for this project.

The following more general assessments of Parkinsonism could be of value simply as a means to demonstrate how severely the patients were affected and whether the experience with them generalizes to other patient populations. They also can serve as a means of stratifying patients so that different prediction algorithms could be constructed for different subgroups of patients. These general assessments of Parkinsonism include:
The UPDRS scale. This is a general assessment of Parkinsonism, having sections covering not only dyskinesia, but also bradykinesia, akinesia and tremor among others. Many studies use subsections of the UPDRS when trying to assess a particular one of the symptoms of Parkinsonism; however this does raise questions of validity of the subparts. The main problem with the UPDRS is that some aspects of it are momentary and some are historical. The same patient can have different UPDRS scores at different times. This limits how useful it would be as a staging of Parkinson's disease.

Hoehn and Yahr staging. This is a 1-5 staging of the severity of Parkinsonism based on level of disability. The expanded UPDRS score (with all 6 sections) actually includes the Hoehn and Yahr staging scale. The Hoehn and Yahr scale is easy to do and does not mix up momentary and historical aspects. The drawback is that it does a very crude staging.

2. Description of Prior Art Including Information Disclosed Under 37 CFR 1.97 and 1.98

Devices used to aid in the assessment of Parkinsonism are either “in laboratory only” devices or ambulatory/wearable devices. Devices that are predominately “in laboratory” devices are useful for monitoring the patient only for brief periods of time. Because one of the overall objects of my invention is to continuously monitor patient movement states over a significant time period, for example several hours or days, only ambulatory/wearable type devices can be employed.

Many different types of sensors have been used to obtain movement data from patients. These include electromyography, ultrasound, radar, laser displacement detectors, mechanical coupling devices, video-based systems, in addition to accelerometers and rotation sensors. However, for the purposes of ambulatory/wearable monitoring, only accelerometric and gyroscopic modalities and perhaps electromyography appear to be feasible. Video monitoring also may be feasible so long as the involved body parts can be maintained in the line of sight of the device.

The standard method for measuring movement in an ambulatory (outside of clinic) setting is accelerometry. Various simplified versions of accelerometers have been studied for movement analysis but have not been found to be as satisfactory as the 3-axis accelerometers utilized in the apparatus which forms a part of the present invention.

Wearable accelerometer devices have been studied for the measurement of movement in Parkinson's patients, but none have been designed in a manner that would be useful for the titration of medications. The data used to create the classification algorithms for those devices generally did not have the continual clinical annotation that would be needed to create a device that could produce a timeline of the patient's movement. Their classification algorithms were generally trained with data derived from structured tasks, and were therefore inappropriate for at-home ambulatory monitoring, which must be able to work in an unstructured environment. Their classification schemes generally address dyskinesia or tremor, but not bradykinesia/hypokinesia, which is clinically important as well as patient subjective self assessment (i.e. patient self reporting diary) which is the most clinically important. The few devices that attempt to detect bradykinesia/hypokinesia do not address them in a way that would be useful for adjusting medications. Furthermore, previous classification schemes generally used simplistic algorithms that could not address the complexity of this problem.

For example, an accelerometer based system has been used for monitoring gait and balance as is disclosed in article entitled “The WAMAS (Wearable Accelerometric Motion Analysis System): Combining Technology Development nd Research n Human Mobility by Eric E. Sabelman et al. However, that system is not used for tracking patient movement over time. It is not used to sense limb movements or to classify dyskinesia or hypokinesia as would be required in medication adjustment for Parkinson's patients.

A similar accelerometer based system appears to have been employed in other reported studies, as set forth in “Reliability and Validity of Accelerometric Gait and Balance” by Eric E. Sabelman et al. and “Advanced Accelerometric Motion Analysis System (Design/Development)”. However, there the apparatus is being used for gait and balance analysis and not for the classification of movement states over time in order to regulate medication for Parkinson's patients.

### BRIEF SUMMARY OF THE INVENTION

It is a prime object of the present invention to provide a method and apparatus for automatically classifying the movement states in Parkinson's disease (including the patient's perception of movement states and severity of symptoms).

It is another object of the present invention to provide a method and apparatus for automatically classifying the movement states in Parkinson's disease for use by the clinician in adjusting the medication of the patient.

It is another object of the present invention to provide a method and apparatus for automatically classifying the movement states in Parkinson's disease that has the ability to utilize information from prior similar patients to make predictions about how a current patient should be scored, without prior scored data from that patient.

It is another object of the present invention to provide a method and apparatus for automatically classifying the movement states in Parkinson's disease that employs a wearable apparatus capable of collecting data from a patient in an unstructured and unencumbered environment, such that the patient can perform normal daily activities while data is being obtained.

It is another object of the present invention to provide a method and apparatus for automatically classifying the movement states in Parkinson's disease that employs a data collection apparatus capable of collecting data relating to patient movement over an extended period of time, preferably greater than 24 hours.

It is another object of the present invention to provide a method and apparatus for automatically classifying the movement states in Parkinson's disease that has the ability to predict the patient's subjective assessment of his or her movement states.

It is another object of the present invention to provide a method and apparatus for automatically classifying the movement states in Parkinson's disease wherein the data model is produced by actual sampling of prior patients without utilizing arbitrary cutoffs or arbitrary algorithms (such arbitrary cutoffs and algorithms can impede the ability of the classifier from properly classifying movement states in new patients and may limit the ability of the classifier to progressively improve predictions when data from progressively more prior patients are used as a basis to make predictions about how a current patient should be scored).

It is another object of the present invention to provide a method and apparatus for automatically classifying the movement states in Parkinson's disease wherein five 3-axis accelerometers worn by the patient are used for data collection.

Those objects are achieved by the present invention which is a monitor for Parkinson's patients developed to record the effect of Parkinson's medication on the patients' movement, enabling Parkinson's medications to be optimally adjusted by clinicians who utilize this information. This requires collecting accurate and reliable information about how Parkinson's patients' movements fluctuate throughout their day.

To accomplish this, a set of wearable sensors (accelerometers) is employed to measure a patient's movements while he or she is performing their normal activities. The data collected by these sensors is then fed to a computer and analyzed by classification algorithms. Ultimately, the output of apparatus is utilized to develop a timeline indicating when the patient had decreased movements, when the patient had fluid movements and when the patient had particular types of abnormal movements (as well as their perceptions of movement state and severity). It is intended that the timeline would be used by the managing clinician to adjust the medications of the patient.

In accordance with one aspect of the present invention, a method is provided for automatically classifying the movement states in a Parkinson's patient. The method begins by creating an algorithm capable of predicting the movement states of a current Parkinson's patient based upon information collected from prior patients. Information as to the movements of the current patient is collected over time. The information collected from the current patient is processed using the prediction algorithm to classify the movement states of the current patient over time. The movement states of the current patient over the given time period obtained in this manner are then recorded.

The movement states prediction algorithm is created by collecting sensor data representative of the movement of prior patients over time utilizing multiple sensors worn by the prior patients. That collected sensor data is converted into a series of data scores representative of the movements of the prior patients over time. At the same time, the prior patients are observed and a series of observation scores representative of the observed movement states of the prior patients over time are assigned. The data scores and observation scores are utilized to create the movement states predicting algorithm.

A series of scores representative of the prior patients' self-assessment of their symptoms (movement states and/or severity) experienced over time are also be assigned. Those self-assessment scores may be used in conjunction with the data scores, instead of, or in addition to the observation scores, to create the movement states predicting algorithm.

The data scores and observation scores are obtained over multiple time segments. Those data scores and the observation scores are utilized to create the movement states prediction algorithm by constructing a “machine learning” program and utilizing the data scores and the observation scores to train the program.

The self-assessment scores are also obtained over multiple time segments. The self-assessment scores may also be used to train the “machine learning” program.

The sensors used to collect the sensor data are accelerometers. The collected sensor data is converted into data scores by first converting the accelerometric data from each accelerometer into a single magnitude for each of multiple points of time in the time segment. A fast Fourier transform is then performed on the magnitudes for multiple time points. The fast Fourier transformation results are then converted to real numbers by obtaining the absolute values thereof. The converted fast Fourier transformation results are integrated over first and second selected frequency ranges. The ratio of the integration results over the selected frequency ranges for each time segment is formed. Covariances for the ratios of the integration results obtained from selected accelerometer pairs for each time segment are calculated. Data scores for each time segment of accelerometer data are assigned based upon the covariances.

The model is obtained by constructing a linear regression model or by constructing a neutral network model.

The accelerometric is converted by converting the accelerometric data from each accelerometer in accordance with the following formula:
Magnitude value=the (positive) square root of (X²+Y²+Z²⁾
wherein X, Y and Z represent the data value obtained for each axis of the accelerometer.

Preferably, the accelerometric data is converted at approximately the sampling rate of the accelerometers. The fast Fourier transform is performed over 800 samples at a time.

Preferably, the first selected frequency range is the sum of values between 0.25 Hz -3 Hz and the second selected frequency range is the sum of values between 4 Hz-6 Hz.

One accelerometer measures hip movement. A second accelerometer measures movement of the right upper extremity. A first covariance is obtained by calculating the covariance of the frequency ratio of the output of the hip movement accelerometer and of the right upper extremity movement accelerometer.

A third accelerometer measures movement of the right lower extremity. A second covariance is obtained by calculating the covariance of the frequency ratio of the output of the hip movement accelerometer and of the right lower extremity movement accelerometer.

A fourth accelerometer measures movement of the left lower extremity. A third covariance is obtained by calculating the covariance of the frequency ratio of the output of the hip movement accelerometer and of the left lower extremity movement accelerometer.

The information from the current patient is collected by collecting the sensor data representative of the movement of the current patient over time utilizing multiple sensors in the form of accelerometers worn by the current patient. The collected sensor data is converted into a series of data scores representative of the movements of the current patient over time. The movement states algorithm is then utilized to create a timeline of the current patient's movement states based upon the current patient's data scores. The timeline is then used by the clinician to manage the medicine of the current patient.

The movement states obtained by the present invention preferably include bradykinesia/hypokinesia and dyskinesia. Those movement states are classified over a time period in which normal activities of the current patient are taking place.

In accordance with another aspect of the present invention, a method is provided for automatically classifying the movement states of patients with Parkinson's disease. The method begins by creating an algorithm capable of predicting the movement states of a current patient, based upon sensed data representative of the movement of the body parts of the current patient, without any prior information about the current patient. Sensed data representative of the movement states of the body parts of the current patient over time is obtained. That sensed data is then processed with the algorithm to provide an output.

A graphical representation of the output over time is created. The graphical representation is used by the clinician to adjust the medication of the patient over time.

In accordance with another aspect of the present invention, a method is provided for automatically classifying the patient's self-assessment of movement states of patients with Parkinson's disease. The method begins by creating an algorithm capable of predicting the patient's self-assessment of movement states of a current patient, based upon sensed data representative of the movement of the body parts of the current patient, without any prior information about the current patient. Sensed data representative of the movement states of the body parts of the current patient over time is obtained. The sensed data is then processed with the algorithm to provide an output.

A graphical representation of the output over time is created. That graphical representation is then used by the clinician to adjust the medication of the patient over time.

The predicted movement states are recorded on a continual basis with no less than one predicted movement state per hour of time that the current patient had movement information collected. Preferably, the period of time in which the predicted movement states are recorded exceeds 2 hours and 30 minutes.

The current patient can participate in normal activities during the time period over which the sensor data is obtained.

The sensed data is collected using a wearable sensor device. The sensor device preferably includes more than one accelerometer attached to different parts of the current patient's body. Preferably, the device includes four or more 3 dimensional accelerometers.

The algorithm is created by selecting prior patients. Information as to the movements over time of the prior patients is collected utilizing sensors. Observational information as to the movement states and/or the patient's self-assessments of symptoms in the prior patients is collected during time intervals corresponding to the time in which the movement states of the prior patient were collected by the sensors.

The algorithm used to process the information is designed to provide increasingly improved predictions for the current patient as data from more prior patients is collected and processed.

In accordance with another aspect of the present invention, apparatus is provided for automatically classifying the movement states in a Parkinson's patient. The apparatus includes means for creating an algorithm capable of predicting the movement states of a current patient based upon information collected from prior patients. Means are provided for collecting information as to the movements of the current patient over time. Means are provided for processing the information collected from the current patient using the prediction algorithm to classify the movement states of the current patient over time. Means are also provided for recording the movement states of the current patient over the given time period.

The means for creating the algorithm includes means for collecting sensor data representative of the movement of prior patients over time utilizing multiple accelerometers worn by the prior patients. Means are provided for converting the collected sensor data into a series of data scores representative of the movements of the prior patients over time. The prior patients are observed and a series of numerical observation scores representative of the observed movement states of the prior patients over time are assigned. Means are provided for utilizing the data scores and observation scores to create the movement states predicting algorithm.

The means for creating the algorithm may also include assigning a series of scores representative of the prior patients' self-assessment of symptoms experienced over time. Means are provided for utilizing the self-assessment scores to create the movement states predicting algorithm.

The data scores and observation scores are obtained over multiple time segments. The means for utilizing the data scores and observation scores to create the movement states prediction algorithm includes means for constructing a “machine learning” program. The data scores and the observation scores are utilized to “train” the “machine learning” program.

The self-assessment scores are also collected over multiple time segments. The self-assessment scores may also be used to train the “machine learning” program.

The sensors preferably take the form of accelerometers. The means for converting the collected sensor data includes means for converting the accelerometric data from each accelerometer into a single magnitude for each of multiple points of time in the time segment. Means are provided for performing a fast Fourier transform on the magnitudes for multiple time points. Means are provided for converting the fast Fourier transformation results to real numbers by obtaining the absolute values thereof. Means are provided for integrating the converted fast Fourier transformation results over first and second selected frequency ranges. Means are provided for forming the ratio of the integration results over the selected frequency ranges for each time segment. Means are provided for obtaining covariances for the ratios of the integration results obtained from selected accelerometer pairs for each time segment. Means are also provided for assigning data scores for each time segment of accelerometer data based upon the covariances.

The means for constructing a model includes means for constructing a linear regression model or means for constructing a neutral network model.

The means for converting the accelerometric data includes means for converting the accelerometric data from each accelerometer in accordance with the following formula:
magnitude value=the (positive) square root of (X²+Y²+Z²⁾
wherein X, Y and Z represent the data value obtained for each axis of the accelerometer.

The means for converting the accelerometric data includes means for converting the accelerometric data at approximately the sampling rate of the accelerometers.

The means for performing a fast Fourier transform includes means for performing the fast Fourier transform over 800 samples at a time.

The first selected frequency range is the sum of values between 0.25 Hz -3 Hz. The second selected frequency range is the sum of values between 4 Hz-6 Hz.

The apparatus includes one accelerometer that measures hip movement and a second accelerometer that measures movement of the right upper extremity. The means for obtaining covariances includes means for obtaining the covariance of the frequency ratio of the output of the hip movement accelerometer and of the right upper extremity movement accelerometer.

The apparatus also includes a third accelerometer that measures movement of the right lower extremity. The means for obtaining covariances includes means for obtaining the covariance of the frequency ratio of the output of the hip movement accelerometer and of the right lower extremity movement accelerometer.

The apparatus also includes a fourth accelerometer measures movement of the left lower extremity and wherein the means for obtaining covariances includes means for obtaining the covariance of the frequency ratio of the output of the hip movement accelerometer and of the left lower extremity movement accelerometer.

The means for collecting information from the current patient includes means for collecting sensor data representative of the movement of the current patient over time utilizing multiple accelerometers worn by the current patient. Means are provided for converting the collected sensor data into a series of data scores representative of the movements of the current patient over time. Means are also provided for utilizing the movement states algorithm to create a timeline of the current patient's movement states based upon the current patient's data scores.

The timeline of the movement states is used to manage the medicine of the current patient. The movement states include bradykinesia/hypokinesia and dyskinesia. Preferably, the movement states are classified over a time period in which normal activities are taking place.

In accordance with another aspect of the present invention, apparatus is provided for automatically classifying the movement states of Parkinsonian patients or patients with Parkinson's disease. The apparatus includes means for creating an algorithm capable of predicting the movement states of a current patient, based upon sensed data representative of the movement of the body parts of the current patient, without any observational information about the current patient. Means are provided for obtaining sensed data representative of the movement states of the body parts of the current patient over time. Means are also provided for processing the sensed data with the algorithm to provide an output.

Means are provided for creating a graphical representation of the output over time. The graphical representation is used to adjust the medication of the patient over time.

In accordance with another aspect of the present invention, apparatus. is provided for automatically classifying the patient's self-assessment of movement states of Parkinsonian patients or patients with Parkinson's disease. The apparatus includes means for creating an algorithm capable of predicting the patient's self-assessment of movement states of a current patient, based upon sensed data representative of the movement of the body parts of the current patient, without any observational information about the current patient. Means are provided for obtaining sensed data representative of the movement states of the body parts of the current patient over time. Means are also provided for processing the sensed data with the algorithm to provide an output.

Means are provided for creating a graphical representation of the output over time. The graphical representation is used to adjust the medication of the patient over time.

Means are provided for recording the predicted movement states on a continual basis, with no less than one predicted movement state per hour of time that the current patient had movement information collected. Preferably, the recording means records the predicted movement states over a time period that exceeds 2 hours and 30 minutes.

The apparatus of the present invention permits the current patient to participate in normal activities during the time period over which the sensor data is obtained.

The means for obtaining sensed data comprises a wearable sensor device. Preferably, the sensor device includes more than one accelerometers attached to different parts of the current patient's body. Most preferably, four or more 3 dimensional accelerometers are used.

The means for creating the algorithm includes means for collecting information as to the movements over time of the prior patients utilizing sensors. Means are also provided for collecting observational information as to the movement states and/or the patient's self-assessments of symptoms in the prior patients during time intervals corresponding to the time in which the movement states of the prior patient were collected by the sensors.

The algorithm is designed to provide increasingly accurate predictions for the current patient as data from more prior patients is collected and processed.

The method of the present invention provides for automatically classifying the movement states in a Parkinson's patient. The method begins by creating an algorithm capable of predicting the movement states of a current Parkinson's patient based upon information collected from prior patients. After the movement states prediction algorithm is created, information as to the movements of the current patient over time is collected by the wearable accelerometers.

That sensor information is processed in the computer, using the previously developed movement states prediction algorithm, to classify the movement states of the current patient over time, in the manner that simulates what the current patient would have entered in his/her diary, had a diary been kept by the current patient or the clinician would have scored, had the patient been observed by a clinician over the time period.

However, the use of my system eliminates the practical problems associated with the necessity for accurate diary keeping and prolonged clinical observation. The classified movement states of the current Parkinson's patient over time obtained from the computer are then recorded for use by the clinician in adjusting the medication of the current patient.

The classification prediction algorithm is created by collecting sensor data representative of the movements of prior patients over time, utilizing multiple sensors in the form of accelerometers worn by the prior patients. The collected sensor data is converted into a series of data scores representative of the movements of the prior patients over time. The movements of the prior patients are observed and a series of numerical observation scores representative of the observed movement states of the prior patients over time are assigned. In addition, a series of numerical subjective scores representative of the prior patients' self-assessment of symptoms experienced over time are assigned. Utilizing those data scores, observation scores and/or self-assessment scores from the prior patients, the movement states prediction algorithm is developed.

The data scores, observation scores and/or self-assessment scores from the prior patients are obtained over multiple time segments. They are used to create the movement states prediction algorithm by “training” the “machine learning” program portion of the algorithm to more accurately predict the movement states of current patients from which data scores are obtained.

The “machine learning” program of the algorithm preferably includes a pre-packaged neural net or linear regression. The training of the “machine learning” program takes place by adjusting the features of the program, for example, the weights of the neural net connections, in accordance with the “training” data that is obtained from prior patients and other settings that are chosen. Further, the input to the “machine learning” program may be refined by changing how the data scores are obtained from the raw data.

The present invention is able to predict, for the current patient, the self-assessment scores that such a patient would generate and observational scores that the clinician would generate based upon observations of the patient. Those scores include the patient's subjective symptom self-assessment (i.e. patient diary), a measure of bradykinesia/hypokinesia as well as a measure of dyskinesia. The present invention is able to make those predictions accurately, without restricting the normal activities of the patient and without observation of the patient by the clinician over extended periods of time.

### BRIEF DESCRIPTION OF THE SEVERAL VIEWS OF DRAWINGS

To those, and to such additional objects that may hereinafter appear, the present invention relates to a method and apparatus for the classification of movement states in Parkinson's disease, as described in detail in the following specification and recited in the annexed claims, taken together with the accompanying drawings in which:

FIG. 1 is a flow chart illustrating the steps employed in creating the prediction algorithm based upon information from prior patients;

FIG. 2 is a flow chart illustrating how the prediction algorithm is used to develop a timeline for use in adjusting the medication of the current patient;

FIG. 3 is a typical timeline that could be created using the output of the present invention for predicted patient diary scores;

FIG. 4 is a flowchart illustrating the steps involved in converting the collected sensor data into data scores;

FIG. 5 is an idealized representation of a patient wearing the apparatus of the present invention and illustrating how the sensor data collected from the patient is transferred to the computer for processing;

FIG. 6 is a schematic representation of the sections of the computer that function to develop the prediction algorithm from the sensor data, observation scores and self-assessment diary scores of prior patients;

FIG. 7 is a schematic representation of the sections of the computer that take the sensor data from the current patient, convert the sensor data into data scores, process the data scores with the prediction algorithm and feed the plotter to form a timeline of the type illustrated in FIG. 3; and

FIG. 8 is a schematic representation of the sections of the computer that convert the collected sensor data into data scores.

### DETAILED DESCRIPTION OF THE INVENTION

Different types of movements in Parkinson's patients tend to have different frequency characteristics. Dyskinesia has been found to be predominately in the lower frequency range (approximately 0.25 Hz-3.5 Hz) and Parkinson's rest tremor at a higher frequency (4-6 Hz). Other types of tremor tend to be in a higher range (essential tremor 7-12 Hz and physiological tremor 8-12 Hz).

Different types of dyskinesia were found to have different frequencies. For example, dystonia has been found to be in the 0.25-1.25 Hz range and chorea in the 1.5-3.25 Hz. Voluntary activity has been found to be in the below 3.3 Hz range with the majority less than 1 Hz (except walking, which was about 2 Hz). Unfortunately, none of these frequency ranges are “hard and fast”. There is also overlap in frequency range between different types of motion.

Rather than a single frequency, an accelerometer actually picks up a spectrum of frequencies. A device might be able to use the predominant frequency in order to better classify what type of movement is occurring. (e.g. voluntary activity versus dyskinesia versus tremor). However, there is more information in the frequency spectrum than simply the peak frequency or mean frequency. The distribution of the frequencies may also help to better classify the type of movement.

The most common technique that has been used to analyze data obtained from wearable sensors has been some form of correlation. This may involve a comparison of the features derived from the device readings with some clinical score that was observed. It may also involve comparison between different readings without comparison to a clinical score, such as might be done to test reliability and validity. Other statistical methods used include analysis of variance, the kappa statistic and linear regression. Hidden Markov models have been used to detect gesture sequences, but not for purposes of detecting free form pathological movements. Neural networks appear to be the only major “machine learning” technique that has been explored.

In order to assess the feasibility of using the system of the present invention to classify on-off range (i.e., hypokinesia and bradykinesia) and dyskinesia in Parkinson's patients, a pilot study was performed on two Parkinson's patients in an observed setting. Two different models of movement classification based on the collected data were trained and tested.

Both patients were determined by their referring neurologist to have motor fluctuations. The patients were observed by a neurologist and were videotaped for later review by the same neurologist. During the study each patient wore the accelerometers for detecting motion. After the observation, the accelerometer output data was downloaded for offline analysis.

The accelerometer apparatus consisted of a series of five 3-axis accelerometers. The range of the accelerometers was from −1 g to +3 g with a resolution of 1/64 g. Samples were taken by the accelerometers at approximately 40 Hz.

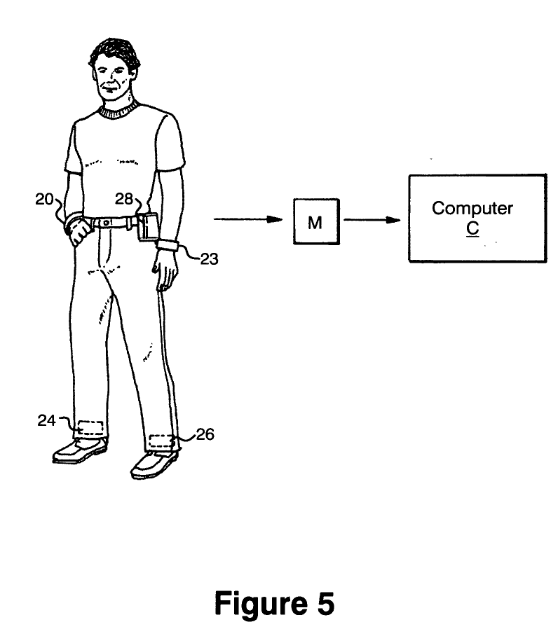

The five accelerometers were attached to the patient using Velcro straps at the following locations: As illustrated in FIG. 5, first and second accelerometers 20, 22 are attached on the dorsum of the right and left arms, respectively, just proximal to the wrist. Third and fourth accelerometers 24, 26 are attached to the right and left leg, respectively, just proximal to the lateral aspect of the ankle. A fifth accelerometer 28 is contained in the main unit, which is attached to the patient's belt proximate the right hip. The main unit is connected to each of the accelerometers by wires (not and contains a data storage device that receives the raw sensor data from each of the accelerometers.

TABLE 1
Scoring scheme used for pilot study
Dyskinesia (chorea only)

| | |
|---|---|
| 0 | none |
| 1 | mild (does not appear to impair patient at all) |
| 2 | moderate (appears to cause mild impairment of activity) |
| 3 | significant (appear to cause moderate impairment |
| | of activity) |
| 4 | severe (appears to cause severe impairment of activity) |

On-Off (a measure of bradykinesia and hypokinesia)

| | |
|---|---|
| 0 | Significantly off |
| 1 | Mildly off |
| 2 | Ambiguous or intermediate |
| 3 | Mildly on |
| 4 | Definitely on |

The data collected from the accelerometers is recorded on a removable chip M. After all of the sensor data is recorded, memory chip M is removed from the main unit and inserted into one of the data input ports of the computer C utilized to process the data.

The observing neurologist queried the patient as to his state and later reviewed the video recording to obtain a synthesis assessment of the state of the patient, referred to as the observation score herein. A 0-4 scoring was used for “on-off” (i.e. bradykinesia and hypokinesia) and a 0-4 scoring for dyskinesia (more specifically chorea). Table 1 shows the scoring scheme that was used.

Notation was made once per minute for the duration of the time the device was recording. If the patient was temporarily off the video or temporarily not observed (e.g. to bathroom), the neurologist would extrapolate the intermediate time points based data known about the surrounding observed time points.

TABLE 2
How the on-off and dyskinesia scales were dichotomized
in the pilot study

| | |
|---|---|
| Low range | High range |

Patient #1

| | | |
|---|---|---|
| On off | <1.5 | >=1.5 |
| Dyskinesia | <2.0 | >=2.0 |

Patient #2

| | | |
|---|---|---|
| On off | <3.5 | >=3.5 |
| Dyskinesia | <2.0 | >=2.0 |

Note:
The text refers to “high level” for on-off as “on” , low level on-off” as “off”. “High level” dyskinesia is referred to as “dyskinetic” and “low level” dyskinesia as “not dyskinetic”

Although a scale of 0-4 was used for dyskinesia and (separately) for on-off, these scorings were then dichotomized to ascertain how well the system was working based on a subjective analysis of what would be a clinically relevant cut off. This was determined based on the range of variation of the patient. Table 2 shows the cutoffs were used for dichotomizing (using the original 0-4 scale):

Data were processed using Java, Matlab, as well as Netlab (for neural network functions). The data were divided into training and test sets for use in a neural

TABLE 3
Size of test sets and training sets for the pilot study

| Patient | Size of Test Set | Size of Training Set | Total |
|---|---|---|---|
| Patient #1 | 124 one minute samples | 186 one minute samples | 310 |
| Patient #2 | 132 one minute samples | 198 one minute samples | 330 |
| Total | 256 one minute samples | 384 one minute samples | 640 |

network (see Table 3). Data for each patient were handled separately. data were divided into one-minute windows and each window was assigned to the training set or test set randomly in a roughly 60:40 ratio. Therefore the test and training sets did not consist of contiguous time periods.

From the accelerometric data, features were derived and used as the basis for neural network and classification tree classification models. Table 4 lists features that were used.

TABLE 4
Features that were extracted from the accelerometry
data and then used as inputs into machine learning
algorithms (pilot study).

| Name | Description |
|---|---|
| R1 | Hip “magnitude” |
| R2 | RUE “magnitude” |
| R3 | LUE “magnitude” |
| R4 | RLE “magnitude” |
| R5 | LLE “magnitude” |
| R6 | RUE and LUE “positional correlation” |
| R7 | RUE and LUE “magnitude correlation” |
| R8* | RUE and LUE “positional mutual information” |
| R9 | Not used |
| R10 | RUE and RLE “positional correlation” |
| R11 | hip and RUE “magnitude correlation” |
| R12 | hip and LUE “magnitude correlation” |

RUE = right upper extremity
LUE = left upper extremity
RLE = right lower extremity
LLE = left lower extremity
For description of “magnitude”, “positional correlation”, “magnitude correlation” and “positional mutual information” please see text.
*This feature was found not help much in classification and because it was very computationally intensive, it was not used in building any of the models

Accelerometric recordings were obtained at a rate of about 40 readings per second. To enter this data into a machine learning program, two possibilities were considered. One way would be to use to data from each individual reading (representing 1/40 of a second) as the input for the machine learning algorithms and the label (e.g. “on-off” and dyskinesia state) for the output. Another approach would be to window the processing in a way that features derived from a whole period of time (e.g. 1 minute) would be used instead of data from only a single reading cycle.

The windowing approach was selected for several reasons. First, if only a single reading were used as the basis for the model, then the classification power of the system would have been very weak. The accelerations at a particular point in time are not likely to be nearly as good a predictor of movement state as those of an entire period of time. This problem could be partially remedied by letting the machine learning program make a prediction based on only a single reading cycle, but then combine these predictions to create a prediction for a whole time window. In that way the prediction for the whole time window would be more powerful because it combines the power of the many individual predictions that were made for each reading cycle.

However, it was not clear as to how best to combine these predictions into one larger prediction. For instance, they could be averaged or multiplied depending on different assumptions. The windowing technique of making the predictions based on the whole window, simplified this problem. Another reason why using the whole window may be better than using only a single reading cycle is that it enables correlations or mutual information between different accelerometers to be generated.

While it is true that the machine learning algorithm may “learn” how different features vary together even if only one time point at a time is taken, that would not take into account the range of values in the time window immediately preceding and following that time point. Time window correlation and mutual information measures adjust for near term variability.

The features that were used as inputs to the computer for the machine learning algorithms are listed in table 4. The gross (measured) acceleration is obtained by simple vector addition (i.e. gross acceleration=(positive) square root of (x²+y²+z²)).

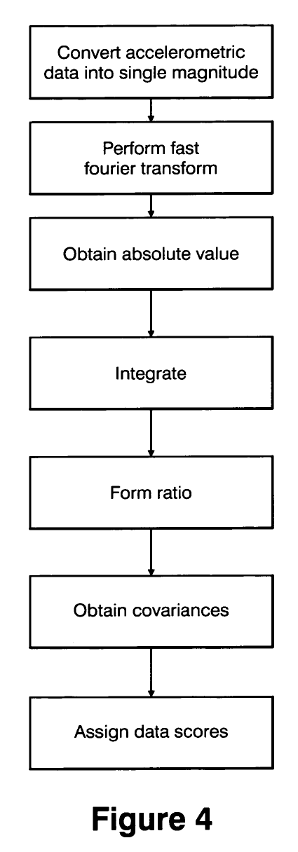

FIGS. 4 and 8 illustrate how the sensor data from the accelerometers is processed to obtain the data scores in the final study. The absolute value of magnitude was used because, clearly averaging the derivative over anything but the shortest period of time would yield zero. Accelerations and decelerations are both measures of movement and were counted equally.

The positional correlation was intended as a measure of common orientation of the limbs involved. Certain activities may be expected to entail different limb orientations. If two limbs have their positional orientations correlated, then it might be expected that they are working together. They way this measure was calculated was as follows: Six factors (the X, Y, and Z axis accelerations of both sensor sites) were correlated with each other in all possible permutations (except that a factor was not correlated with itself). The mean of these 15 correlations was called the “positional correlation”.

The magnitude correlation is actually the correlation of the derivative of measured acceleration over the time window involved (1 minute).

Certain repeated positions might signify certain activities (e.g. walking). However, it might be expected that some position of the two sensors are common in a particular activity, but may not be detectable by simple correlation. For this reason, positional mutual information was used.

Positional mutual information was calculated in a manner similar to “positional correlation”, however, instead of correlations, mutual information was used as it in theory might be more appropriate than simple correlation. The process to

TABLE 5
Method for calculating mutual information using
accelerometry data from two separate
accelerometers (pilot study)

| | |
|---|---|
| 1. | Take the accelerometry data of the 2 sensors |
| | in question for the time window in |
| | question. Call those 2 strings of data |
| | vector X and vector Y. |
| 2. | Discretize the vectors X and Y as follows: |
| | replace the values for acceleration with |
| | a number indicating what quartile (or decile) |
| | that value belongs to relative to the |
| | other values for acceleration in that same |
| | vector (in this project both quartiles and |
| | deciles were tested). Call these new discretized |
| | vectors X_{d} and Y_{d}. (Quartiles and |
| | deciles are labeled starting from zero) |
| 3. | Create a vector W by combining X_{d} and |
| | Y_{d} as follows: W(t), the element of the |
| | vector W that corresponds to a particular timepoint |
| | t, is set to X_{d} (t) * (# of |
| | possible values of Y_{d}) + Y_{d} (t). |
| 4. | Mutual information is calculated using Shannon's |
| | entropy: MI = entropy(X_{d}) + |
| | entropy(Y_{d}) − entropy(W) |

calculate the mutual information of 2 sensors (for a particular time window) is described in Table 5.

Rather than minute-by-minute dyskinesia as a target output, a 10-minute moving average was used. This produced better results on the training set, presumably because dyskinesia varies a lot over the very short term and may be missed using smaller windows.

To implement the neural network part of the experiment, Netlab (an extension of Matlab) was used. Coding was done in Matlab, R and Java. The implemented neural network used a single hidden layer of neurons. Hidden nodes used a tanh activation function and the (single) output neuron used a logistic function.

The feature space (and neural network parameters) was explored using 5-fold cross-validation on the training set. Features for the test set were chosen based on results of the cross-validation on the training set. Table 6 shows the features that were selected.

TABLE 6
Features used for neural network models in the pilot study

Patient #1 “on-off” (Model #1)
Inputs:
Hip absolute of derivative of magnitude for the window (R1)*
RUE absolute of derivative of magnitude for the window (R2)
RLE absolute of derivative of magnitude for the window (R4)
LLE absolute of derivative of magnitude for the window (R5)
RUE/RLE positional correlation for the window (R10)
Output:
The average on-off rating for the 1 minute window
Neural net:
6 hidden nodes and 100 iterations
Patient #2 “on-off” (model #2)
Inputs (same as patient #1):
Hip absolute of derivative of magnitude for the window (R1)+
RUE absolute of derivative of magnitude for the window (R2)
RLE absolute of derivative of magnitude for the window (R4)
LLE absolute of derivative of magnitude for the window (R5)
RUE/RLE positional correlation for the window (R10)
Output (same as patient #1):
The average on-off rating for the 1 minute window
Neural net (same as patient #1):
6 hidden nodes and 100 iterations
Patient #1 dyskinesia (model #3)
Inputs:
Hip absolute of derivative of magnitude for the window (R1)
RUE absolute of derivative of magnitude for the window (R2)
Hip and RUE magnitude correlation for window (R11)
Output:
A ten minute moving average of dyskinesia
Neural net:
Hidden nodes 6 iterations 100
Patient #2 dyskinesia (model #4)
Inputs:
RUE absolute of derivative of magnitude for the window (R2)
RLE absolute of derivative of magnitude for the window (R4)
Output:
A ten minute moving average of dyskinesia
Neural net:
Hidden nodes 4, iterations 200
*Note: names of features in parentheses refer to the features listed in Table 4
+Hip accelerometer yielded missing data for section of recording. For this part, this feature was assigned a value of zero

In order to assess the calibration of the neural network classification model, Hosmer-Lemeshow c-hat and h-hat goodness-of-fit statistics were obtained. For Hosmer-Lemeshow c-hat, the samples were divided into quartiles as in table 7:

TABLE 7
Ranges used by Hosmer-Lemeshow c-hat (pilot study)

| Range # | Expected value |
|---|---|
| 1 | <25 percentile |
| 2 | >=25 and <50 percentile |
| 3 | >=50 and <75 percentile |
| 4 | >=75 percentile |

For the Hosmer-Lemeshow h-hat, the samples were divided into 4 ranges as in table 8.

TABLE 8
Ranges used by Hosmer-Lemeshow h-hat (pilot study)

| Range # | Expected value |
|---|---|
| 1 | <0.25 |
| 2 | >=0.25 and <0.5 |
| 3 | >=0.5 and <0.75 |
| 4 | >0.75 |

The results of the Hosmer-Lemeshow tests for each of the neural network models are own in Table 9.

TABLE 9
Results (pilot study) of Hosmer-Lemeshow test for neural network
models (using the test set only)

| | | |
|---|---|---|
| Model | p-value | Degrees of freedom |

Hosmer-Lemeshow c-hat

| | | |
|---|---|---|
| Patient #1 on-off | 0.8154 | 7 |
| Patient #2 on-off | 0.07559 | 7 |
| Patient #1 dyskinesia | 0.2438 | 7 |
| Patient #2 dyskinesia | 0.593 | 7 |

Hosmer-Lemeshow h-hat

| | | |
|---|---|---|
| Patient #1 on-off | Not calculable | N/A |
| Patient #2 on-off | 0.9864 | 3 |
| Patient #1 dyskinesia | 0.468 | 5 |
| Patient #2 dyskinesia | 0.7504 | 7 |

After assessing the results of the pilot study, it was decided to utilize certain items of information that had not been collected in the pilot study in the final study. It was decided to use both physician-based observation scoring and subjective patient symptom assessment scoring based on patient diaries. They were used to create separate classification models. Since the patient subjective self-assessment diary is the commonly used scheme against which the results obtained by the system of the present invention could be compared, an attempt was made to classify movements based on patient diaries.

Further, in the final study, more standardized metrics were utilized. Baseline Hoehn & Yahr and MMSE scores were employed in order to gauge generalizability.

One of the major goals of the final study was to demonstrate that in the present invention the prediction algorithm would be able to predict patient subjective symptom self-assessment scores. It is not intuitive that this should be accomplishable because the patient self-assessment diary is based on how the patient feels, not on how he/she moves and how the patient feels would not appear to be something that can be ascertained simply by observing the patient as it often differs from how observers score the patient.

Another major goal of the final study was to demonstrate that in the present invention accurate classification could be done on a current patient even without the use of training data from that patient. This could be a difficult problem because patients vary so much from each other. For instance, in the pilot study, the cutoff above which patient #1 was a score of 1.5, whereas the cutoff used for patient #2 was 3.5 (see table 2). Those cutoffs were based on clinical observations, which is information that the classifying algorithms will not have access to. Therefore, it would be difficult for the algorithms to classify, if the cutoffs for dichotomization are not known.

There are other problems too. For instance, the value of features may vary widely across patients. An algorithm which relies on fixed values of individual features to differentiate classes is likely to make errors. Algorithms such as logistic or linear regression or neural networks which use combinations of features should be more robust.

Accordingly, arbitrary cutoffs were not used to dichotomize data. Instead a regression was performed and then a series of cutoffs were applied. The effectiveness of the algorithms was judged by how well they classified using all the dichotomization cutoffs. In this way, no clinical knowledge would be needed in order to choose the right cutoff for the patient and a general assessment could be obtained of how well the algorithms performed at all the possible classification tasks.

Cutoffs were based on percentile for the particular patient were employed because using cutoffs based on fixed numbers does not take into account what is considered a high score or a low score for that particular patient. Using given percentiles as cutoffs for the patient in question helps remedy this problem.

This system, however, was not applied to cutoffs used for dichotomizing diary scores. Diary scores are different because they inherently take into account what is high or low for that particular patient. That is because in diary scoring, the patient is asked to subjectively assess how they are doing and that would be based on the patient specific thresholds.

Regression algorithms were used because they seemed most appropriate to assess goodness of fit using error measures based on deviation of the predicted value from the actual value. These include mean squared error, mean absolute error and the R2 statistic. The standard error functions were to the dichotomizations

The basic analysis was done on very short segments of accelerometry data. The results of the basic analyses were aggregated over the entire 10-minute of analysis. In the pilot study, features were derived from processing the entire 1-minute period as a whole. Observing the patients, it was noticed that many actions occurred more in fits and starts than as continuous activity. This could lead to small burst of perhaps irrelevant activity obscuring more important subtler actions that are present for a large fraction of the time, but are not as dramatic. Using small segments to do basic analyses on and then aggregating these analyses (e.g. by taking covariance) makes short bursts of activity less relevant.

In addition, frequency analysis was used. This is because of the importance of frequency as noted in the literature.

All of the patients selected for the final study were determined to have the diagnosis of Parkinson's disease and to have significant fluctuations in their movements, either fluctuations between bradykinesia and eukinesia (“on” vs. “off”) and/or fluctuations in their degree of dyskinesia.

Five new patients participated in the final study. Additionally, the two patients from the pilot study were also included in the analysis. Since some types of data were only collected in the final study, some aspects of the analysis could only be performed on the patients from the final part of the study.

All patients were tested using a Folstein mini-mental status examination (a common screening test for dementia) and required to have at least a score of 24/30. Additionally, a Hoehn and Yahr staging was performed on each patient to gauge the level of their Parkinsonism.

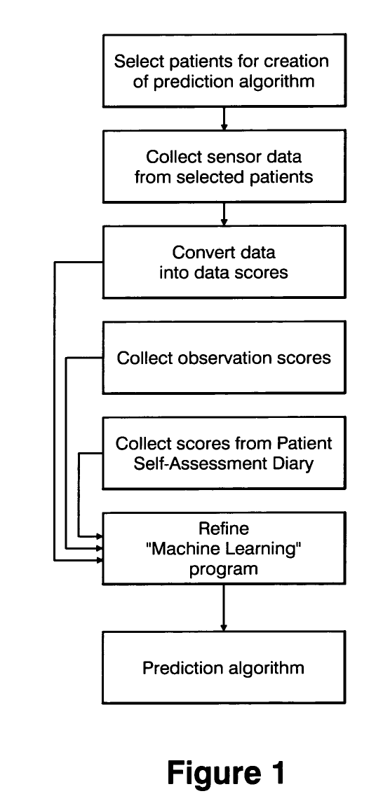

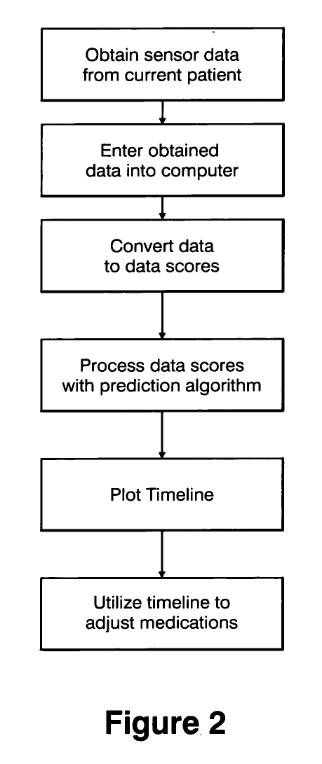

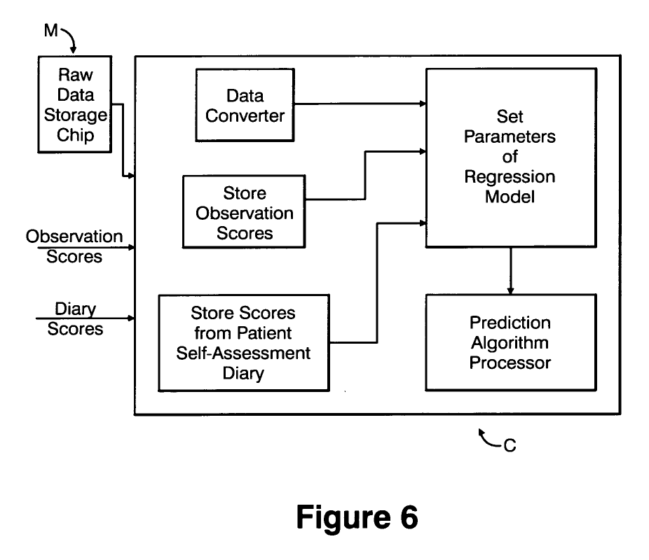

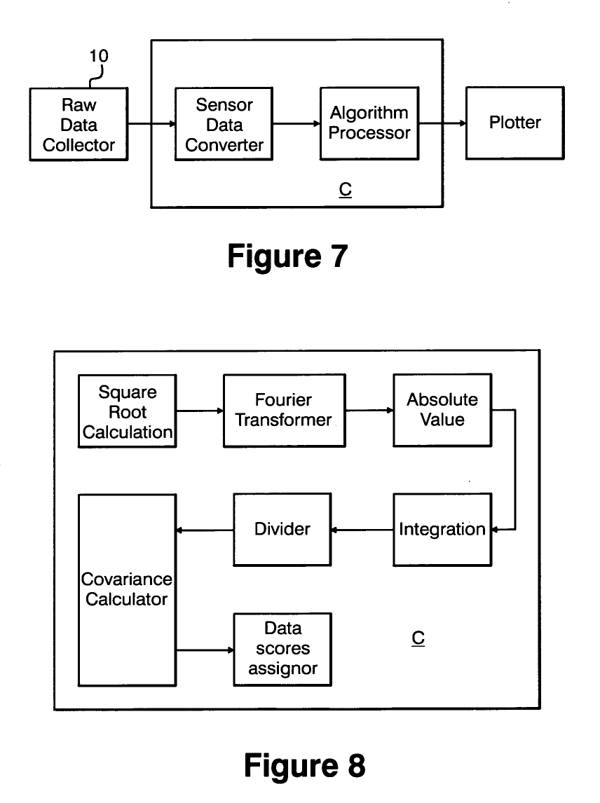

FIGS. 1 and 6 illustrate how the movement states prediction algorithm was developed for use in the final study. All patients in the final study were observed by a neurologist and videotaped for later review by the same neurologist. Clinical observations and the scorings were recorded by the neurologist every 10 minutes.

Additionally, patients were asked to complete a diary every 30 minutes noting their symptom self-assessment, including of state of their movements and the severity thereof. Patient self-assessment scores were assigned to the diary entries to represent the assessment of the patients.

Simultaneous to the clinical observations and the patient self-assessment scorings, each patient wore five accelerometers, as illustrated schematically in FIG. 5, identical to those described in the pilot study. As in the pilot study, the accelerometers 20 through 28 were placed distally on each extremity as well as on the right hip (attached to belt or trousers). At a later time, all patients had their video recordings reviewed and a final determination of the observation scorings was assigned by the clinician.

The two patients in the pilot study did not have this systematic diary information recorded. Additionally, since the scoring scheme done differed in time for the two parts of the study, the videotapes of the two pilot study patients needed to be reviewed and re-scored.

Tables 12, 13 and 14 contain list of the clinical observation scores that were obtained on the study patients.

TABLE 12
Final study clinical scores based on observations (recorded every 10
minutes) obtained on the five patients of the final study.

| Number | Label name | Description |
|---|---|---|
| 1 | AIMS_overall | Level of dyskinesia overall for the whole body, based on |
| | | AIMS² (0 = none, 1 = minimal, 2 = mild, 3 = mild, 4 = severe) |
| 2 | AIMS_UE | Level of dyskinesia for the upper extremities, based on |
| | | AIMS² (0 = none, 1 = minimal, 2 = mild, 3 = mild, 4 = severe) |
| 3 | AIMS_LE | Level of dyskinesia for the lower extremities, based on |
| | | AIMS² (0 = none, 1 = minimal, 2 = mild, 3 = mild, 4 = severe) |
| 4 | AIMS_trunk | Level of dyskinesia for the trunk, based on AIMS² |
| | | (0 = none, 1 = minimal, 2 = mild, 3 = mild, 4 = severe) |
| 5 | Dyskinesia_old | Dyskinesia scoring scheme used in the pilot study |
| | | (0 = none, 1 = mild , does not appear to impair patient at all, |
| | | 2 = moderate, appears to cause mild impairment of |
| | | activity, 3 = significant, appears to cause moderate |
| | | impairment of activity, 4 = severe, appears to cause |
| | | severe impairment of activity) |
| 6 | BBH | Body bradykinesia and hypokinesia (item #31 of the |
| | | Unified Parkinson Disease Rating Scale²⁷). Combining |
| | | slowness, hesitancy, decreased arm swing, small |
| | | amplitude and poverty of movement in general score as |
| | | follows: (0 = none, 1 = minimal slowness giving movement |
| | | a deliberate character; could be normal for some |
| | | persons. Possibly reduced amplitude, 2 = Mild degree of |
| | | slowness and poverty of movement which is definitely |
| | | abnormal. Alternatively, some reduced amplitude, |
| | | 3 = Moderate slowness, poverty or small amplitude of |
| | | movement, 4 = Marked slowness, poverty or small |
| | | amplitude of movement) |
| 7 | On_off | Scoring scheme used in the pilot study to gauge “on ” |
| | | vs. “off” state (0 = significantly off, 1 = mildly off, |
| | | 2 = ambiguous or intermediate, 3 = mildly on, 4 = definitely |
| | | on) |
| 8 | Tremor_RUE | Rest tremor score for right upper extremity (based on |
| | | item #20 of the UPDRS²⁷). (0 = absent, 1 = slight and |
| | | infrequently present, 2 = mild in amplitude and persistent |
| | | or moderate in amplitude but only intermittently present, |
| | | 3 = moderate in amplitude and present most of the time, |
| | | 4 = Marked in amplitude and present most of the time. |
| 9 | Tremor_LUE | Rest tremor score for left upper extremity (scored as |
| | | above). |
| 10 | Tremor_RLE | Rest tremor score for right lower extremity (scored as |
| | | above). |
| 11 | Tremor_LLE | Rest tremor score for left lower extremity (scored as |
| | | above). |

TABLE 13
Final study patient diary scores (recorded every 30 minutes)
obtained on the five patients of the final study.

| Number | Label name | Description |
|---|---|---|
| 1 | Diary | Patient notes how the patient believes he |
| | | or she has been over the past 30 minutes |
| | | (0 = asleep, 1 = off, 2 = on without |
| | | dyskinesia, 3 = on with non-troublesome |
| | | dyskinesia, 4 = on with troublesome |
| | | dyskinesia) |

TABLE 14
Pilot study clinical scores based on observations (recorded
every 10 minutes) obtained from the five patients in the
final study as well as from the two pilot patients.

| Number | Label name | Description |
|---|---|---|
| 1 | On_off | Same as #7 above |
| 2 | BBH | Same as #6 above |
| 3 | Dyskinesia_old | Same as #5 above |
| 4 | AIMS_overall | Same as #1 above |

All accelerometry data was off-loaded from the device's removable flash card M and processed off-line in a computer. . C language code was used to convert the recordings into ASCII format. Subsequent data processing and analyses were performed with the help of custom-written code in Java (Sun Microsystems), Matlab (Matlab 12, by Mathworks), SAS(SAS institute) and Neurosolutions (by Neurodimension). SAS was used for linear regression and Neurosolutions was used for neural networks.

Because of the limited number of patients in the study, it was felt that there would not be enough patients for a true validation set. Without a true validation set, it would not be possible to adjust the features and parameters used in the linear regression and neural network models after the analysis has begun. Adjusting the features and parameters for the models in order to optimize the results, in the absence of a true validation set would likely lead to results that are unreliable and likely better than they would be in reality.

In order to avoid this problem, all the features that would be used were determined before analysis. When constructing the models, only default settings were used (no adjustment of parameters). The features that were used in all the models were chosen based on experience from the pilot study, as well as from information obtained from the literature (results on the pilot study patients were later compared with those of the final study patients to determine whether using information from the pilot study to design the analysis led to inappropriately better results for the pilot study patients).

Each of the five 3-axis accelerometers employed consisted of two 2-axis accelerometers aligned perpendicularly to each other. Two of the four readings were for the same axis and were therefore averaged together (mean) to form a single reading. As indicted in FIGS. 4 and 8, the readings from the three axes were combined to form a single reading corresponding to magnitude of the overall vector (using the Pythagorean equation: magnitude=the (positive) square root of ((x²+y²+z²)).

The magnitude value obtained was subject to a fast Fourier transform (FFT). The FFTs were obtained over 800 samples at a time. Since the device sampled at slightly less than 40 Hz, this corresponded to slightly more than 20 seconds of recordings.

The FFT values were then converted to real (non-imaginary) values by obtaining the absolute value. An integration was performed to obtain the sum of all values (area under the curve) corresponding to the following frequency ranges:
1. Sum of values 0.25 Hz -3 Hz
2. Sum of values 4 Hz -6 Hz

The ratio of the two sums was calculated. Since the unit of analysis was the 10-minute time period (corresponding to a single set of clinical scores), those ratios were combined to obtain a single value for the whole 10 minute time period. This was achieved by obtaining the covariance of this ratio in one accelerometer versus that of another accelerometer.

There were ten possible pairs of accelerometers for which covariance could be obtained. However, based on the results of the pilot study patients, only three were chosen: covariance of frequency ratio between hip and right upper extremity; covariance of frequency ratio between hip and right lower extremity; and covariance of frequency ratio between hip and left lower extremity.

Linear regression was performed by SAS version 8 (using the “analyst” program). Neural network models were constructed using Neurosolutions. All default parameters were used, including the following:
1. Model: multilayered perceptron
2. 1 hidden layer
3. regression
4. tanh transfer function
5. 1000 epochs

The five final study patients had accelerometry recordings for a total of 13 hours, 38 minutes and 43 seconds. The break down is shown in table 15.

TABLE 15
Final study accelerometry recordings

| Patient | Accelerometry recordings |
|---|---|
| #1 | One sequence of 2:39:09 in length |
| #2 | Two sequences. One 1:54:35 in length another 1:10:34 in length |
| #3 | Two sequences. One 30:11 in length another 1:50:00 in length |
| #4 | One sequence 2:30:22 in length |
| #5 | One sequence 3:03:52 in length |

TABLE 16
Time blocks by patient

| Patient | Number of 10-minute blocks |
|---|---|
| Patient #1 | 17 |
| Patient #2 | 20 |
| Patient #3 | 12 |
| Patient #4 | 16 |
| Patient #5 | 19 |
| Pilot Patient #1 | 32 |
| Pilot Patient #2 | 15 |

The labels had the attributes as shown in table 17. General information about the final study patients is shown in table 18.

TABLE 17
Mean and Standard deviation of clinical labels for each patient

| | | BBH | AIMS_overall | AIMS_overall | | Diary |
|---|---|---|---|---|---|---|
| Patient | BBH (mean) | (std) | (mean) | (std) | Diary (mean) | (std) |
| #1 | 0.94 | 0.6587 | 0.47 | 0.7174 | 1.35 | 0.7859 |
| #2 | 1.30 | 1.4179 | 1.55 | 0.9987 | 2.25 | 0.9105 |
| #3 | 0.67 | 0.4924 | 1.25 | 1.4848 | 2.50 | 0.9045 |
| #4 | 1.06 | 0.9287 | 0.19 | 0.5439 | 2.00 | 0.8165 |
| #5 | 0.89 | 1.1496 | 1.37 | 1.1648 | 2.21 | 0.7133 |
| Pilot 1 | 1.50 | 1.3912 | 0.91 | 0.9625 | N/A | N/A |
| Pilot 2 | 1.47 | 1.3558 | 1.33 | 1.4960 | N/A | N/A |

(std: standard deviation from mean)

TABLE 18
General features of the final study patients

| Patient | Age | Gender | Hoehn & Yahr | Handedness |
|---|---|---|---|---|
| #1 | 62 | Male | Stage 3 | Right |
| #2 | 62 | Female | Stage 4 | Right |
| #3 | 77 | Female | Stage 4 | Right |
| #4 | 52 | Male | Stage 3 | Right |
| #5 | 62 | Male | Stage 3 | Right |

Because of the small amount of observed tremor and because most dyskinesia appeared to be generalized, the analysis was focused on only the three target variables, as shown in table 19.

TABLE 19
Clinical labels used in analysis

1. body bradykinesia and hypokinesia (BBH)
2. AIMS overall (AIMS_overall)
3. diary (Diary)

Since the diary was only recorded every three time blocks, the patient's scoring was applied to all three previous time blocks (i.e. the past 30 minutes). This was appropriate because, when completing the diary, the patients were instructed to assess how they were “over the last 30 minutes.

The two scores initially used in the pilot study (on_off and dyskinesia_old) attempted to measure the same characteristics as target variables #1 and #2 above and were therefore felt to be redundant.

For both linear regression and neural network (regression), a leave-1-out method was used to compile a series of training and test sets. For instance, a model would be constructed using 6 patients and would then be tested on the patient not used in constructing the model. In the case of the diary, the model would be constructed based on only 4 patients and then tested on the remaining patient. Since different patients had different numbers of time blocks, the training set for each model was obtained by randomly resampling the time blocks of each patient so that each patient would end up with 50 time blocks to be used to construct the model. This way, patients with more data would not be over-represented in the models.

It was considered to be important that the time relation of target values be taken into account. This could have been done using a hidden Markov model, but a very simple technique was used instead. The predicted value for each (10 minute) time block was substituted by the median value of the current time block, the previous time block and the time block that follows. The intention of this was to screen out predictions that were outliers and were not in line with the surrounding predictions.

The overall results were obtained as shown in tables 20 and 21.

TABLE 20
Linear regression results overview

| | Average | Average c- | Mean absolute | |
|---|---|---|---|---|
| Target | correlation | index | error | R² |
| BBH | 0.6441 | 0.8219 | 0.7905 | 0.1220 |
| AIMS (overall) | 0.5289 | 0.7552 | 0.8301 | 0.2730 |
| Diary | 0.6143 | 0.8799 | 0.6853 | 0.2262 |
| | | (0.8815) | | |

TABLE 21
Neural network results overview

| | Average | Average c- | Mean absolute | |
|---|---|---|---|---|
| Target | correlation | index | error | R² |
| BBH | 0.6356 | 0.8043 | 0.8203 | 0.1885 |
| AIMS (overall) | 0.4495 | 0.6398 | 0.7717 | 0.3133 |
| Diary | 0.4125 | 0.7374 | 0.6851 | 0.1563 |
| | | (0.7243) | | |

The average correlation was obtained by obtaining the correlation of the measured target value with the predicted target value for each of the patients. Those correlations were then averaged (mean) to obtain a single value for “average correlation”.

C-index (equivalent to the area under the receiver operator characteristics curve) requires a dichotomous variable in order to be calculated. Clearly, the c-indices would be different if different cut-points would be used to dichotomize the variables. Here, several different cut-points were used and c-index results for the different cut-points were averaged for each patient. Then the average of all the patients was calculated (i.e. the average c-index).

Since it was felt that the absolute value of the AIMS score or BBH score for a particular patient would not be as relevant as whether it is low or high for that particular patient, cutoffs were obtained based on percentiles for that patient. Nine cut-offs were obtained (10 percentile, 20 percentile, 30 percentile, 40 percentile, 50 percentile, 60 percentile, 70 percentile, 80 percentile, 90 percentile).

In contrast to the AIMS and BBH scores, the actual value of the diary score should be relevant clinically because it is a direct measure of how the hypokinesia, bradykinesia and dyskinesia affects the individual. Therefore, cut-offs were not obtained using percentiles for that particular patient, but rather were obtained by fixed cutoffs (0.5, 1.5, 2.5, 3.5). The average c-index obtained using the percentile method is included in parentheses for comparison.

The mean absolute error was obtained by obtaining the mean absolute error for each patient and averaging it over all patients.

R2 is a statistic used to assess goodness-of-fit. A value of 1 corresponds to perfect prediction of the target value. A value of zero corresponds to a fit that is no better than simply guessing that the value is the same as the mean (of the data that were used to build the model).

More detailed statistics on all models are shown in tables 22-33.

TABLE 22
Linear regression BBH model: c-indices using different percentile cutoffs

| Per- | | | | | | | |
|---|---|---|---|---|---|---|---|
| cen- | | | | | | | |
| tile | | | | | | | |
| as | | | | | | | |
| cutoff | Pat#1 | Pat#2 | Pat#3 | Pat#4 | Pat#5 | Pilot1 | Pilot2 |
| 10 | N/A | N/A | N/A | N/A | N/A | N/A | N/A |
| 20 | N/A | N/A | N/A | N/A | N/A | N/A | N/A |
| 30 | 0.8750 | N/A | N/A | N/A | N/A | N/A | N/A |
| 40 | 0.8750 | 0.8132 | 1.0000 | 0.5818 | N/A | 0.4909 | 0.8796 |
| 50 | 0.8750 | 0.8132 | 1.0000 | 0.5818 | N/A | 0.4909 | 0.9018 |
| 60 | 0.8750 | 0.8132 | 1.0000 | 0.5818 | 0.8056 | 0.6412 | 0.9018 |
| 70 | 0.8750 | 0.8690 | 1.0000 | 0.8909 | 0.8056 | 0.6412 | 0.7000 |
| 80 | 0.8750 | 0.9531 | 1.0000 | 0.8909 | 0.9214 | 0.6412 | 0.7000 |
| 90 | 0.8571 | 0.9412 | 1.0000 | 0.8909 | 0.9375 | 0.7704 | 0.7000 |
| Mean | 0.8724 | 0.8672 | 1.0000 | 0.7364 | 0.8675 | 0.6126 | 0.7972 |

TABLE 23
Linear regression AIMS overall model: c-indices using different
percentile cutoffs

| Per- | | | | | | | |
|---|---|---|---|---|---|---|---|
| cen- | | | | | | | |
| tile | | | | | | | |
| as | | | | | | | |
| cutoff | Pat#1 | Pat#2 | Pat#3 | Pat#4 | Pat#5 | Pilot1 | Pilot2 |
| 10 | N/A | N/A | N/A | N/A | N/A | N/A | N/A |
| 20 | N/A | 0.8672 | N/A | N/A | N/A | N/A | N/A |
| 30 | N/A | 0.8672 | N/A | N/A | N/A | N/A | N/A |
| 40 | N/A | 0.8229 | N/A | N/A | 0.7679 | N/A | N/A |
| 50 | N/A | 0.8229 | 0.9000 | N/A | 0.6818 | 0.5992 | N/A |
| 60 | N/A | 0.8229 | 0.9000 | N/A | 0.6818 | 0.5992 | 0.9722 |
| 70 | 0.6136 | 0.8229 | 1.0000 | N/A | 0.6818 | 0.5992 | 0.9722 |
| 80 | 0.6136 | 0.8229 | 1.0000 | N/A | 0.6818 | 0.6594 | 0.9722 |
| 90 | 1.0000 | 0.6569 | 1.0000 | 0.4643 | 0.7708 | 0.6594 | 0.9722 |
| Mean | 0.7424 | 0.8132 | 0.9600 | 0.4643 | 0.7110 | 0.6233 | 0.9722 |

TABLE 24
Linear regression Diary model: c-indices (using fixed cutoffs)

| Patient | 0.5 cutoff | 1.5 cutoff | 2.5 cutoff | 3.5 cutoff | Mean |
|---|---|---|---|---|---|
| Pat #1 | N/A | 0.9672 | 0.9672 | N/A | 0.9672 |
| Pat #2 | N/A | 1.0000 | 0.8788 | N/A | 0.9394 |
| Pat #3 | N/A | N/A | 1.0000 | 1.0000 | 1.0000 |
| Pat #4 | N/A | 0.9273 | 0.4364 | N/A | 0.6818 |
| Pat #5 | N/A | 0.9375 | 0.6667 | N/A | 0.8021 |
| Mean | N/A | 0.9602 | 0.7916 | 1.0000 | |

(note: there is no diary information for the 2 pilot patients)

TABLE 25
Neural Network regression BBH model c-indices using different
percentile cutoffs

| Per- | | | | | | | |
|---|---|---|---|---|---|---|---|
| cen- | | | | | | | |
| tile | | | | | | | |
| as | | | | | | | |
| cutoff | Pat#1 | Pat#2 | Pat#3 | Pat#4 | Pat#5 | Pilot1 | Pilot2 |
| 10 | N/A | N/A | N/A | N/A | N/A | N/A | N/A |
| 20 | N/A | N/A | N/A | N/A | N/A | N/A | N/A |
| 30 | 0.8269 | N/A | N/A | N/A | N/A | 0.6208 | N/A |
| 40 | 0.8269 | 0.7363 | 1.0000 | 0.5455 | N/A | 0.6208 | 0.8796 |
| 50 | 0.8269 | 0.7363 | 1.0000 | 0.5455 | N/A | 0.7285 | 0.9018 |
| 60 | 0.8269 | 0.7363 | 1.0000 | 0.5455 | 0.8111 | 0.7285 | 0.9018 |
| 70 | 0.8269 | 0.8214 | 1.0000 | 0.7636 | 0.8111 | 0.7285 | 0.7000 |
| 80 | 0.8269 | 0.9063 | 1.0000 | 0.7636 | 0.9214 | 0.7971 | 0.7000 |
| 90 | 0.6667 | 0.8529 | 1.0000 | 0.7636 | 0.8750 | 0.8269 | 0.7000 |
| Mean | 0.8040 | 0.7982 | 1.0000 | 0.6545 | 0.8547 | 0.7216 | 0.7972 |

TABLE 26
Neural Network regression AIMS overall model: c-indices using
different percentile cutoffs

| Per- | | | | | | | |
|---|---|---|---|---|---|---|---|
| cen- | | | | | | | |
| tile | | | | | | | |
| as | | | | | | | |
| cutoff | Pat#1 | Pat#2 | Pat#3 | Pat#4 | Pat#5 | Pilot1 | Pilot2 |
| 10 | N/A | N/A | N/A | N/A | N/A | N/A | N/A |
| 20 | N/A | 0.7578 | N/A | N/A | N/A | N/A | N/A |
| 30 | N/A | 0.7578 | N/A | N/A | N/A | N/A | N/A |
| 40 | N/A | 0.7031 | N/A | N/A | 0.8333 | N/A | N/A |
| 50 | N/A | 0.7031 | 0.4143 | N/A | 0.7670 | 0.6619 | N/A |
| 60 | N/A | 0.7031 | 0.4143 | N/A | 0.7670 | 0.6619 | 0.9722 |
| 70 | 0.4394 | 0.7031 | 0.7500 | N/A | 0.7670 | 0.7386 | 0.9722 |
| 80 | 0.4394 | 0.7031 | 0.7500 | N/A | 0.7670 | 0.7386 | 0.9722 |
| 90 | 1.0000 | 0.5490 | 0.7000 | 0.0714 | 0.8854 | 0.7386 | 0.9722 |
| Mean | 0.6263 | 0.6975 | 0.6057 | 0.0714 | 0.7978 | 0.7080 | 0.9722 |

TABLE 27
Neural Network regression Diary model: c-indices (using fixed cutoffs)

| Patient | 0.5 cutoff | 1.5 cutoff | 2.5 cutoff | 3.5 cutoff | Mean |
|---|---|---|---|---|---|
| Pat #1 | N/A | 0.9808 | 0.9808 | N/A | 0.9808 |
| Pat #2 | N/A | 0.9400 | 0.8385 | N/A | 0.8893 |
| Pat #3 | N/A | N/A | 1.0000 | 1.0000 | 1.0000 |
| Pat #4 | N/A | 0.1250 | 0.3750 | N/A | 0.2500 |
| Pat #5 | N/A | 0.5208 | 0.6131 | N/A | 0.5670 |
| Mean | N/A | 0.6417 | 0.7615 | 1.0000 | |

(note: there is no diary information for the 2 pilot patients)

TABLE 28
Linear Regression: BBH model

| Patient | Mean squared error | Mean absolute error |
|---|---|---|
| Pat #1 | 0.2774 | 0.4107 |
| Pat #2 | 1.2878 | 0.8110 |
| Pat #3 | 0.2639 | 0.4412 |
| Pat #4 | 0.7444 | 0.6874 |
| Pat #5 | 0.9631 | 0.8474 |
| Pilot1 | 2.4257 | 1.2618 |
| Pilot2 | 1.4208 | 1.0737 |

TABLE 29
Linear Regression AIMS overall model

| Patient | Mean squared error | Mean absolute error |
|---|---|---|
| Pat #1 | 0.4576 | 0.6457 |
| Pat #2 | 0.7960 | 0.7346 |
| Pat #3 | 1.4052 | 0.8851 |
| Pat #4 | 0.4138 | 0.5921 |
| Pat #5 | 1.4147 | 1.0292 |
| Pilot1 | 0.8526 | 0.7733 |
| Pilot2 | 1.8036 | 1.1510 |

TABLE 30
Linear Regression Diary model

| Patient | Mean squared error | Mean absolute error |
|---|---|---|
| Pat #1 | 1.0761 | 1.0289 |
| Pat #2 | 0.4006 | 0.5505 |
| Pat #3 | 0.7095 | 0.6059 |
| Pat #4 | 0.6863 | 0.6972 |
| Pat #5 | 0.4477 | 0.5439 |

TABLE 31
Neural Network BBH model

| Patient | Mean squared error | Mean absolute error |
|---|---|---|
| Pat #1 | 0.3768 | 0.5291 |
| Pat #2 | 1.2784 | 0.8767 |
| Pat #3 | 0.3592 | 0.5889 |
| Pat #4 | 1.1745 | 0.8822 |
| Pat #5 | 0.7146 | 0.7284 |
| Pilot1 | 2.1900 | 1.1556 |
| Pilot2 | 1.2043 | 0.9811 |

TABLE 32
Neural Network AIMS_overall model

| Patient | Mean squared error | Mean absolute error |
|---|---|---|
| Pat #1 | 0.2743 | 0.5202 |
| Pat #2 | 1.1487 | 0.7806 |
| Pat #3 | 1.9626 | 1.1259 |
| Pat #4 | 0.3299 | 0.4908 |
| Pat #5 | 0.8402 | 0.7094 |
| Pilot1 | 0.6586 | 0.6954 |
| Pilot2 | 1.8442 | 1.0794 |

TABLE 33
Neural Network Diary model

| Patient | Mean squared error | Mean absolute error |
|---|---|---|
| Pat #1 | 1.0977 | 1.0338 |
| Pat #2 | 0.3993 | 0.5208 |
| Pat #3 | 0.4238 | 0.4628 |
| Pat #4 | 0.8618 | 0.8178 |
| Pat #5 | 0.5453 | 0.5904 |

It appears that in this study the linear regression performed somewhat better than neural network models. This may have been a result of the inability to adjust the parameters of the neural network in order to optimize results, which was a necessary restriction to avoid over-fitting.

Linear regression appeared to perform reasonably well for both the BBH (body bradykinesia/hypokinesia) model and the Diary model (average c-indices of 0.8219 and 0.8719, respectively). Evaluation data shows a quite remarkable performance of linear regression in classifying the diary score.

Clinically, the most important (i.e. relevant) information for management of Parkinsonism is:
1. Whether the patient feels on or off; and
2. Whether the patient has troublesome dyskinesia or not.

The clinician observations are generally felt to be less relevant. In addition, non-troublesome dyskinesias are not nearly as relevant as troublesome dyskinesias. Those two most relevant pieces of information are discerned nearly perfectly by the linear regression model (for diary). The model is able to discern off (diary scores 0,1) from on (diary scores 2,3,4) with a c-index of 0.9602 and to discriminate troublesome dyskinesias (diary score 4) from all others with a c-index of 1.

The AIMS model appeared to perform less well than all the rest (average c-index 0.7552). In the pilot study, dyskinesia had actually been easier to predict than on_off. The reason why the models performed less well across patients is not clear.

C-indices were chosen for dichotomized data rather than mean absolute error, mean squared error or the R² statistic as the main determinant of success or failure of a model because such dichotomization will likely be necessary in order to produce a report that the managing clinician could readily understand. As can be seen in tables 20 and 21, there is generally an inverse relationship between average c-indices and mean absolute error (with the exception of the neural network model for BBH). The R² statistic, which uses the squared errors in its calculation, does not increase with the better models as might have been expected. This is likely because using the square of errors makes it particularly susceptible to a few predicted values that are far off from their target values. This would also be true of the mean absolute error, but to a lesser degree. When the data are going to be dichotomized anyway, these error measures would not be that relevant.

Since no true validation set could be constructed, a cross validation approach was used, but all the features and parameters used in model construction were fixed before analysis was performed. Since the pilot patients were included in most of the analysis and the lessons learned from the pilot study were used in constructing models, it could be argued that the pilot study patients may receive and unfair advantage by having the model specifically tailored to them. While this cannot be entirely dismissed, it is possible to demonstrate that the models did not perform grossly better on those patients.

Table 34 below does not show a dramatic difference between the pilot study patients and all the patients as a whole. In some models they performed slightly better and in some models slightly worse.

TABLE 34
Performance of pilot study patients as compared with all patients
in the study

| | | | | | Mean |
|---|---|---|---|---|---|
| | | | | | average c- |
| | Pilot | Pilot | Mean | | index of |
| | patient | Patient | average c- | Mean | all patients |
| | #1 | #2 | index of | average c- | excluding |
| | average | average | pilot | index of all | pilot study |
| Model | c-index | c-index | patients | 7 patients | patients |
| BBH (linear | 0.6126 | 0.7972 | 0.7094 | 0.8219 | 0.8687 |
| regression) | | | | | |
| AIMS_overall | 0.6233 | 0.9722 | 0.7977 | 0.7552 | 0.7382 |
| (linear | | | | | |
| regression) | | | | | |
| BBH (neural | 0.7216 | 0.7972 | 0.7594 | 0.8043 | 0.8223 |
| network) | | | | | |
| AIMS_overall | 0.7080 | 0.9722 | 0.8401 | 0.6398 | 0.5597 |
| (neural | | | | | |
| network) | | | | | |
| Mean | 0.6664 | 0.8847 | 0.7766 | 0.7553 | 0.7472 |

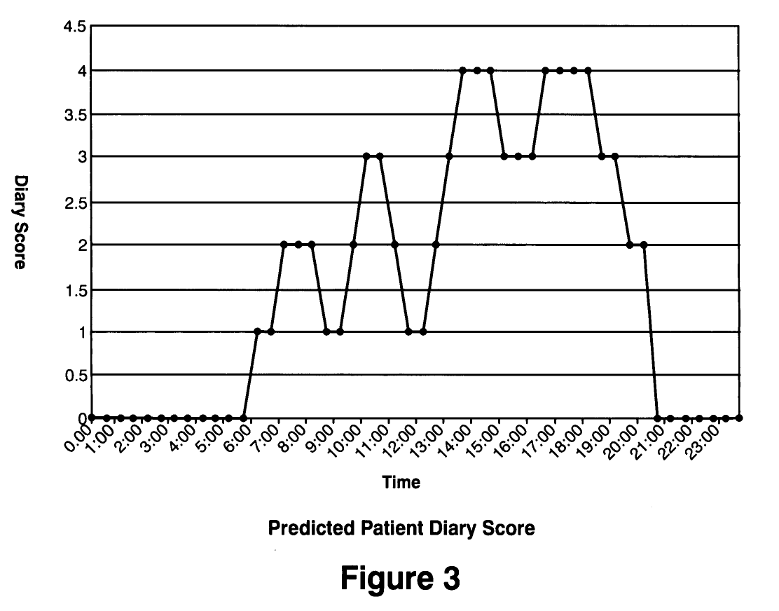

A typical timeline of predicted patient diary score is illustrated in FIG. 3. Similar plots can be used for dyskinesia or bradykinesia/hypokinesia. However, the scale of the y-axis would have to be adjusted to a 5 point scale.

The results that were obtained in this study appear to be quite promising although a classifier constructed using far more patients than were used here would yield even more accurate models. If higher sensitivity and specificity would be desired, readily available data about the patient might be integrated into the models to yield even better results. For instance, age, gender, handedness, and Parkinson stage are easily available and may help fine-tune the models for specific patients.

It will now be appreciated that the present invention relates to a method and apparatus for the classification of movement states in Parkinson's patients that is capable of providing a timeline of movement states which can be used by the physician to adjust the medication of the patient in order to better control the disease.

The system provides the ability to make predictions about how a patient should be scored clinically without having any prior score data for that patient. Because wearable accelerometers are employed to collect movement data from the patient, the patient can function in an unstructured and unencumbered environment such that data can be collected while normal daily activities are taking place. Further, data can be collected over a relatively large time frame, on the order of hours, preferably more that 24 hours.

The present invention provides the ability to predict the subjective diary entries of the patient. It provides output as a numerical score or score range that is clinically useful for the clinician for medication adjustment.

The data model produced is based upon actual sampling of prior patients. No arbitrary cutoffs or arbitrary algorithms are utilized. Such arbitrary cutoffs and algorithms can impede the ability of the classifier from properly classifying movement states in new patients and may limit the ability of the classifier to progressively improve predictions when data from progressively more prior patients are used as a basis to make predictions about how a current patient should be scored.

While only a single preferred embodiment of the present invention has been disclosed for purposes of illustration, it is obvious that many variations and modifications could be made thereto. It is intended to cover all of those variations and modifications that fall within the scope of the present invention, as defined by the following claims:

1. A method for automatically classifying the movement states in a Parkinson's patient, the method comprising the steps of:
creating an algorithm capable of predicting the movement states of a current patient based upon information collected from prior patients;
collecting information as to the movements of the current patient over time; and
processing the information collected from the current patient using the prediction algorithm to classify the movement states of the current patient over time; and
recording the movement states of the current patient over the given time period.

2. The method of claim 1 wherein the step of creating the algorithm comprises the steps of:
collecting sensor data representative of the movement of prior patients over time utilizing multiple sensors worn by the prior patients;
converting the collected sensor data into a series of data scores representative of the movements of the prior patients over time;
observing the prior patients and assigning a series of observation scores representative of the observed movement states of the prior patients over time; and
utilizing the data scores and observation scores to create the movement states predicting algorithm.

3. The method of claim 1 wherein the step of creating the algorithm comprises the following steps:
collecting sensor data representative of the movement of prior patients over time utilizing multiple sensors worn by the prior patients;
converting the collected sensor data into a series of data scores representative of the movements of the prior patients over time;
assigning a series of scores representative of the prior patients' self-assessment of the symptoms experienced over time; and
utilizing the data scores and self-assessment scores to create the movement states predicting algorithm.

4. The method claim 2 wherein the data scores and observation scores are obtained over multiple time segments and wherein the step of utilizing the data scores and observation scores to create the movement states prediction algorithm comprises the steps of:
constructing a “machine learning” program; and
utilizing the data scores and the observation scores to train the program.

5. The method claim 3 wherein the data scores and self-assessment scores are obtained over multiple time segments and wherein the step of utilizing the data scores and self-assessment scores to create the movement states prediction algorithm comprises the steps of:
constructing a “machine learning” program; and
utilizing the data scores and the self-assessment scores to train the of the program.

6. The method of claim 2 wherein the step of converting the collected sensor data comprises the steps of:
converting the data from each sensor into a single magnitude for each of multiple points of time in the time segment;
performing a fast Fourier transform on the magnitudes for multiple time points;
converting the fast Fourier transformation results to real numbers by obtaining the absolute values thereof;
integrating the converted fast Fourier transformation results over first and second selected frequency ranges;
forming the ratio of the integration results over the selected frequency ranges for each time segment;
obtaining covariances for the ratios of the integration results obtained from selected accelerometer pairs for each time segment; and
assigning data scores for each time segment of accelerometer data based upon the covariances.

7. The method of claim 3 wherein the step of converting the collected sensor data comprises the steps of:
converting the data from each sensor into a single magnitude for each of multiple points of time in the time segment;
performing a fast Fourier transform on the magnitudes for multiple time points;
converting the fast Fourier transformation results to real numbers by obtaining the absolute values thereof;
integrating the converted fast Fourier transformation results over first and second selected frequency ranges;
forming the ratio of the integration results over the selected frequency ranges for each time segment;
obtaining covariances for the ratios of the integration results obtained from selected accelerometer pairs for each time segment; and
assigning data scores for each time segment of accelerometer data based upon the covariances.

8. The method of claim 4 wherein the step of constructing a “machine learning” program comprises the step of constructing a linear regression model.

9. The method of claim 5 wherein the step of constructing a “machine learning” program comprises the step of constructing a neutral network model.

10. The method of claim 6 wherein the step of converting the sensed data comprises the step of converting the data from each sensor in accordance with the following formula:
magnitude value=the (positive) square root of (X²+Y²+Z²⁾
wherein X, Y and Z represent the data value obtained for each axis of the accelerometer.

11. The method of claim 7 wherein the step of converting the data comprises the step of converting the accelerometric data from each sensor in accordance with the following formula:
magnitude value=the (positive) square root of (X²+Y²+Z²⁾
wherein X, Y and Z represent the data value obtained for each axis of the accelerometer.

12. The method of claim 6 wherein the sensors are accelerometers and wherein the step of converting the data comprises converting the data at approximately the sampling rate of the accelerometers.

13. The method of claim 7 wherein the sensors are accelerometers and wherein step of converting the data comprises converting the data at approximately the sampling rate of the accelerometers.

14. The method of claim 6 wherein the step of performing a fast Fourier transform comprises the step of performing the fast Fourier transform over 800 samples at a time.

15. The method of claim 7 wherein the step of performing a fast Fourier transform comprises the step of performing the fast Fourier transform over 800 samples at a time.

16. The method of claim 6 wherein the first selected frequency range is the sum of values between 0.25 Hz-3 Hz.

17. The method of claim 7 wherein the first selected frequency range is the sum of values between 0.25 Hz-3 Hz

18. The method of claim 16 wherein the second selected frequency range is the sum of values between 4 Hz-6 Hz.

19. The method of claim 17 wherein the second selected frequency range is the sum of values between 4 Hz-6 Hz.

20. The method of claim 6 wherein the sensors are accelerometers and one accelerometer measures hip movement and another accelerometer measures movement of the right upper extremity and wherein the step of obtaining covariances comprises the step obtaining the covariance of the frequency ratio of the output of the hip movement accelerometer and of the right upper extremity movement accelerometer.

21. The method of claim 7 wherein the sensors are accelerometers and wherein one accelerometer measures hip movement and another accelerometer measures movement of the right upper extremity and wherein the step of obtaining covariances comprises the step obtaining the covariance of the frequency ratio of the output of the hip movement accelerometer and of the right upper extremity movement accelerometer.

22. The method of claim 6 wherein the sensors are accelerometers and wherein one accelerometer measures hip movement and another accelerometer measures movement of the right lower extremity and wherein the step of obtaining covariances comprises the step of obtaining the covariant of the frequency ratio of the output of the hip movement accelerometer and of the right lower extremity movement accelerometer.

23. The method of claim 7 wherein the sensors are accelerometers and wherein one accelerometer measures hip movement and another accelerometer measures movement of the right lower extremity and wherein the step of obtaining covariances comprises the step of obtaining the covariant of the frequency ratio of the output of the hip movement accelerometer and of the right lower extremity movement accelerometer.

24. The method of claim 6 wherein the sensors are accelerometers and wherein one accelerometer measures hip movement and another accelerometer measures movement of the left lower extremity and wherein the step of obtaining covariances comprises obtaining the covariance of the frequency ratio of the output of the hip movement accelerometer and of the left lower extremity movement accelerometer.

25. The method of claim 7 wherein the sensors are accelerometers and wherein one accelerometer measures hip movement and another accelerometer measures movement of the left lower extremity and wherein the step of obtaining covariances comprises obtaining the covariance of the frequency ratio of the output of the hip movement accelerometer and of the left lower extremity movement accelerometer.

26. The method of claim 1 wherein the step of collecting information from the current patient comprises the steps of:
collecting the sensor data representative of the movement of the current patient over time utilizing multiple sensors worn by the current patient;
converting the collected sensor data into a series of data scores representative of the movements of the current patient over time;
utilizing the movement states algorithm to create a timeline of the current patient's movement states based upon the current patient's data scores.

27. The method of claim 26 further comprising the step of utilizing the timeline to manage the medicine of the current patient.

28. The method of claim 1 wherein the movement states comprise bradykinesia/hypokinesia.

29. The method of claim 1 wherein the movement states comprise dyskinesia.

30. The method of claim 1 wherein the movement states are classified over a time period in which the patient can participate in normal activities.

31. A method for automatically classifying the movement states of patients with Parkinson's disease comprising the steps of:
creating an algorithm capable of predicting the movement states of a current patient based upon sensed data representative of the movement of the body parts of the current patient without ongoing observational or self-assessment data from the current patient;
obtaining sensed data representative of the movement states of the body parts of the current patient over time; and
processing the sensed data with the algorithm to provide an output.

32. The method of claim 31 further comprising the steps of:
creating a graphical representation of the output over time; and
utilizing the graphical representation to adjust the medication of the patient over time.

33. A method for automatically classifying the patient's self-assessment of movement states of patients with Parkinson's disease comprising the steps of:
creating an algorithm capable of predicting the patient's self-assessment of movement states of a current patient based upon sensed data representative of the movement of the body parts of the current patient without ongoing observational or self-assessment data from the current patient;
obtaining sensed data representative of the movement states of the body parts of the current patient over time; and
processing the sensed data with the algorithm to provide an output.

34. The method of claim 33 further comprising the steps of:
creating a graphical representation of the output over time; and
utilizing the graphical representation to adjust the medication of the patient over time.

35. The method of claim 31 wherein the predicted movement states are recorded on a continual basis with no less than one predicted movement state per hour of time that the current patient had movement information collected.

36. The method of claim 33 wherein the predicted movement states are recorded on a continual basis with no less than one predicted movement state per hour of time that the current patient had movement information collected.

37. The method of claim 35 wherein the period of time in which the predicted movement states are recorded exceeds 2 hours and 30 minutes.

38. The method of claim 36 wherein the period of time in which the predicted self-assessment of movement states are recorded exceeds 2 hours and 30 minutes.

39. The method of claim 31 wherein the current patient can participate in normal activities during the time period over which the sensor data is obtained.

40. The method of claim 33 wherein the current patient can participate in normal activities during the time period over which the sensor data is obtained.

41. The method of claim 31 wherein the step of obtaining sensed data comprises the step of collecting sensed data using a wearable device.

42. The method of claim 33 wherein the step of obtaining sensed data comprises the step of collecting sensed data using a wearable device.

43. The method of claim 41 wherein the step of collecting sensed data comprises the step of wearing more than one accelerometer attached to different parts of the current patient's body.

44. The method of claim 42 wherein the step of collecting sensed data comprises the step of wearing more than one accelerometer attached to different parts of the current patient's body.

45. The method of claim 41 wherein the step of collecting sensed data comprises the step of wearing four or more 3 dimensional accelerometers.

46. The method of claim 42 wherein the step of collecting sensed data comprises the step of wearing four or more 3 dimensional accelerometers.

47. The method of claim 31 wherein the step of creating the algorithm comprises the steps of:
selecting prior patients;
collecting information as to the movements over time of the prior patients utilizing sensors; and
collecting observational information as to the movement states and/or the patient's self-assessments of movement states in the prior patients during time intervals corresponding to the time in which the movement states of the prior patient were collected by the sensors.

48. The method of claim 33 wherein the step of creating the algorithm comprises the steps of:
selecting prior patients;
collecting information as to the movements over time of the prior patients utilizing sensors; and
collecting observational information as to the movement states and/or the patient's self-assessments of symptoms in the prior patients during time intervals corresponding to the time in which the movement states of the prior patient were collected by the sensors.

49. The method of claim 31 wherein the step of creating an algorithm comprises the step of creating an algorithm that provides increasingly improved predictions for the current patient as data from more prior patients is collected and processed.

50. The method of claim 33 wherein the step of creating an algorithm comprises the step of creating an algorithm that provides increasingly improved predictions for the current patient as data from more prior patients is collected and processed.

51. Apparatus for automatically classifying the movement states in a Parkinson's patient comprising means for creating an algorithm capable of predicting the movement states of a current Parkinson's patient based upon information collected from prior patients; means for collecting information as to the movements of the current patient over time; means for processing the information collected from the current patient using the prediction algorithm to classify the movement states of the current patient over time; and means for recording the movement states of the current Parkinson's patient over the given time period.

52. The apparatus of claim 51 wherein the means for creating the algorithm comprises means for collecting sensor data representative of the movement of prior patients over time utilizing multiple sensors worn by the prior patients; means for converting the collected sensor data into a series of data scores representative of the movements of the prior patients over time; wherein the prior patients are observed and a series of observation scores representative of the observed movement states of the prior patients over time are assigned; and means for utilizing the data scores and observation scores to create the movement states predicting algorithm.

53. The apparatus of claim 51 wherein the means for creating the algorithm comprises means for collecting sensor data representative of the movement of prior patients over time utilizing multiple sensors worn by the prior patients; means for converting the collected sensor data into a series of data scores representative of the movements of the prior patients over time; wherein a series of scores representative of the prior patients' self-assessment of the symptoms experienced over time are assigned; and means for utilizing the data scores and self-assessment scores to create the movement states predicting algorithm.

54. The apparatus claim 52 wherein the data scores and observation scores are assigned over multiple time segments and wherein the means for utilizing the data scores and observation scores to create the movement states prediction algorithm comprises means for constructing a “machine learning” program; and means for utilizing the data scores and the observation scores to train the program.

55. The apparatus claim 53 wherein the data scores and self-assessment scores are assigned over multiple time segments and wherein the means for utilizing the data scores and self-assessment scores to create the movement states prediction algorithm comprises means for constructing a “machine learning” program; and means for utilizing the data scores and the self-assessment scores to train the program

56. The apparatus of claim 52 wherein the means for converting the collected sensor data comprises means for converting the data from each sensor into a single magnitude for each of multiple points of time in the time segment; means for performing a fast Fourier transform on the magnitudes for multiple time points; means for converting the fast Fourier transformation results to real numbers by obtaining the absolute values thereof; means for integrating the converted fast Fourier transformation results over first and second selected frequency ranges; means for forming the ratio of the integration results over the selected frequency ranges for each time segment; means for obtaining covariances for the ratios of the integration results obtained from selected accelerometer pairs for each time segment; and means for assigning data scores for each time segment of accelerometer data based upon the covariances.

57. The apparatus of claim 53 wherein the means for converting the collected sensor data comprises means for converting the data from each sensor into a single magnitude for each of multiple points of time in the time segment; means for performing a fast Fourier transform on the magnitudes for multiple time points; means for converting the fast Fourier transformation results to real numbers by obtaining the absolute values thereof; means for integrating the converted fast Fourier transformation results over first and second selected frequency ranges; means for forming the ratio of the integration results over the selected frequency ranges for each time segment; means for obtaining covariances for the ratios of the integration results obtained from selected accelerometer pairs for each time segment; and means for assigning data scores for each time segment of accelerometer data based upon the covariances.

58. The apparatus of claim 54 wherein the means for constructing a “machine learning” program comprises means for constructing a linear regression model.

59. The apparatus of claim 55 wherein the means for constructing a “machine learning” program comprises the step of constructing a neutral network model.

60. The apparatus of claim 56 wherein the sensors are accelerometers and wherein the means for converting the data comprises means for converting the accelerometric data from each accelerometer in accordance with the following formula:
magnitude value=the (positive) square root of (X²+Y²+Z²⁾
wherein X, Y and Z represent the data value obtained for each axis of the accelerometer.

61. The apparatus of claim 57 wherein the sensors are accelerometers and wherein the means for converting the data comprises means for converting the accelerometric data from each accelerometer in accordance with the following formula:
magnitude value=the (positive) square root of (X²+Y²+Z²⁾
wherein X, Y and Z represent the data value obtained for each axis of the accelerometer.

62. The apparatus of claim 56 wherein the sensors are accelerometers and wherein the means for converting the data comprises means for converting the accelerometric data at approximately the sampling rate of the accelerometers.

63. The apparatus of claim 57 wherein the sensors are accelerometers and wherein the means for converting the accelerometric data comprises means for converting the accelerometric data at approximately the sampling rate of the accelerometers.

64. The apparatus of claim 56 wherein the means for performing a fast Fourier transform comprises means for performing the fast Fourier transform over 800 samples at a time.

65. The apparatus of claim 57 wherein the means for performing a fast Fourier transform comprises means for performing the fast Fourier transform over 800 samples at a time.

66. The apparatus of claim 56 wherein the first selected frequency range is the sum of values between 0.25 Hz-3 Hz.

67. The apparatus of claim 57 wherein the first selected frequency range is the sum of values between 0.25 Hz-3 Hz

68. The apparatus of claim 66 wherein the second selected frequency range is the sum of values between 4 Hz-6 Hz.

69. The apparatus of claim 67 wherein the second selected frequency range is the sum of values between 4 Hz-6 Hz.

70. The apparatus of claim 56 wherein the sensors are accelerometers and wherein one accelerometer measures hip movement and another accelerometer measures movement of the upper right extremity and wherein the means for obtaining covariances comprises means for obtaining the covariance of the frequency ratio of the output of the hip movement accelerometer and of the upper right extremity movement accelerometer.

71. The apparatus of claim 57 wherein the sensors are accelerometers and wherein one accelerometer measures hip movement and another accelerometer measures movement of the upper right extremity and wherein the means for obtaining covariances comprises means for obtaining the covariance of the frequency ratio of the output of the hip movement accelerometer and of the upper right extremity movement accelerometer.

72. The apparatus of claim 56 wherein the sensors are accelerometers and wherein one accelerometer measures hip movement and another accelerometer measures movement of the lower right extremity and wherein the means for obtaining covariances comprises means for obtaining the covariant of the frequency ratio of the output of the hip movement accelerometer and of the lower right extremity movement accelerometer.

73. The apparatus of claim 57 wherein the sensors are accelerometers and wherein one accelerometer measures hip movement and another accelerometer measures movement of the lower right extremity and wherein the means for obtaining covariances comprises means for obtaining the covariant of the frequency ratio of the output of the hip movement accelerometer and of the lower right extremity movement accelerometer.

74. The apparatus of claim 56 wherein the sensors are accelerometers and wherein one accelerometer measures hip movement and another accelerometer measures movement of the lower left extremity and wherein the means for obtaining covariances comprises means for obtaining the covariance of the frequency ratio of the output of the hip movement accelerometer and of the lower left extremity movement accelerometer.

75. The apparatus of claim 57 wherein the sensors are accelerometers and wherein one accelerometer measures hip movement and another accelerometer measures movement of the lower left extremity and wherein the means for obtaining covariances comprises means for obtaining the covariance of the frequency ratio of the output of the hip movement accelerometer and of the lower left extremity movement accelerometer.

76. The apparatus of claim 51 wherein the means for collecting information from the current patient comprises means for collecting the sensor data representative of the movement of the current patient over time utilizing multiple accelerometers worn by the current patient; means for converting the collected sensor data into a series of data scores representative of the movements of the current patient over time; and means for utilizing the movement states algorithm to create a timeline of the current patient's movement states based upon the current patient's data scores.

77. The apparatus of claim 76 wherein the timeline is used to manage the medicine of the current patient.

78. The apparatus of claim 51 wherein the movement states comprise bradykinesia/hypokinesia.

79. The apparatus of claim 51 wherein the movement states comprise dyskinesia.

80. The apparatus of claim 51 wherein the movement states are classified over a time period in which normal activities are taking place.

81. Apparatus for automatically classifying the movement states of patients with Parkinson's disease comprising means for creating an algorithm capable of predicting the movement states of a current patient based upon sensed data representative of the movement of the body parts of the current patient without any prior information about the current patient; means for obtaining sensed data representative of the movement states of the body parts of the current patient over time; and means for processing the sensed data with the algorithm to provide an output.

82. The apparatus of claim 81 further comprising means for creating a graphical representation of the output over time, wherein the graphical representation is used to adjust the medication of the patient over time.

83. Apparatus for automatically classifying the patient's self-assessment of movement states of patients with Parkinson's disease comprising means for creating an algorithm capable of predicting the self-assessment of movement states of a current patient based upon sensed data representative of the movement of the body parts of the current patient without any prior information about the current patient; means for obtaining sensed data representative of the movement states of the body parts of the current patient over time; and means for processing the sensed data with the algorithm to provide an output.

84. The apparatus of claim 83 further comprising means for creating a graphical representation of the output over time, wherein the graphical representation is used to adjust the medication of the patient over time.

85. The apparatus of claim 81 further comprising means for recoding the predicted movement states on a continual basis with no less than one predicted movement state per hour of time that the current patient had movement information collected.

86. The apparatus of claim 83 wherein the recording means records the predicted movement states on a continual basis with no less than one predicted movement state per hour of time that the current patient had movement information collected.

87. The apparatus of claim 85 wherein the recording means records the predicted movement states over a time period that exceeds 2 hours and 30 minutes.

88. The apparatus of claim 86 wherein the recording means records the predicted self-assessment of movement states over a period of time that exceeds 2 hours and 30 minutes.

89. The apparatus of claim 81 wherein the current patient can participate in normal activities during the time period over which the sensor data is obtained.

90. The apparatus of claim 83 wherein the current patient can participate in normal activities during the time period over which the sensor data is obtained.

91. The apparatus of claim 81 wherein the means for obtaining sensed data comprises a wearable device.

92. The apparatus of claim 83 wherein the means for obtaining sensed data comprises a wearable device.

93. The apparatus of claim 81 wherein the means for collecting sensed data comprises more than one accelerometer attached to different parts of the current patient's body.

94. The apparatus of claim 83 wherein the means for collecting sensed data comprises more than one accelerometer attached to different parts of the current patient's body.

95. The apparatus of claim 81 wherein the means for collecting sensed data comprises four or more 3 dimensional accelerometers.

96. The apparatus of claim 83 wherein the means for collecting sense data comprises the four or more 3 dimensional accelerometers.

97. The apparatus of claim 81 wherein the means for creating the algorithm comprises means for collecting information as to the movements over time of the prior patients utilizing sensors; wherein observational information as to the movement states and/or the patient's self-assessments of movement states in the prior patients is collected during time intervals corresponding to the time in which the movement states of the prior patient were collected by the sensors.

98. The apparatus of claim 83 wherein the means for creating the algorithm comprises means for collecting information as to the movements over time of the prior patients utilizing sensors; wherein observational information as to the movement states and/or the patient's self-assessments of movement states in the prior patients is collected during time intervals corresponding to the time in which the movement states of the prior patient were collected by the sensors.

99. The apparatus of claim 81 wherein the means for creating an algorithm comprises means for creating an algorithm that provides increasingly improved predictions for the current patient as data from more prior patients is collected and processed.

100. The apparatus of claim 83 wherein the means for creating an algorithm comprises means for creating an algorithm that provides increasingly improved predictions for the current patient as data from more prior patients is collected and processed.
